(19) United States
(12) Patent Application Publication
(10) Pub. No.: US 2015/0329461 A1
(43) Pub. Date: Nov. 19, 2015

(54) METHODS FOR SEPARATING MIXTURES OF FATTY ACIDS

(71) Applicant: UNIVERSITY OF IOWA RESEARCH FOUNDATION, Iowa City, IA (US)

(72) Inventors: Ned B. Bowden, Iowa City, IA (US); Abhinaba Gupta, Iowa City, IA (US)

(73) Assignee: UNIVERSITY OF IOWA RESEARCH FOUNDATION, Iowa City, IA (US)

(21) Appl. No.: 14/649,862

(22) PCT Filed: Jan. 4, 2013
(86) PCT No.: PCT/US13/20339

§ 371 Date: Jun. 4, 2015

Related U.S. Application Data
(60) Provisional application No. 61/734,249, filed on Dec. 6, 2012.

(51) Int. Cl. C07C 51/47

(52) U.S. Cl. CPC C07C 51/47

(57) ABSTRACT

The invention provides methods for separating mixtures of two or more fatty acids.

### RELATED APPLICATION

This application claims priority to U.S. Provisional Patent Application No. 61/734,249, filed 6 Dec. 2012, the entirety of which is incorporated herein by reference.

### GOVERNMENT FUNDING

This invention was made with government support under CHE-0848162 and CHE-1213325 awarded by the National Science Foundation. The government has certain rights in the invention.

### BACKGROUND OF THE INVENTION

Over 140 million tons of vegetable oils are produced in the United States each year and approximately 96% of the production of these oils are used for food for humans, feed for animals, and biodiesel. This number is expected to greatly increase if biodiesels from algal are produced in significant quantities. These oils are triesters of glycerol (HOCH₂CHOHCH₂OH) and three fatty acids; each fatty acid contains 16, 18, 20, or 22 carbons and zero, one, or more carbon-carbon double bonds (FIG. 1). Vegetable oils are found in over 20,000 different foods, and they are the source of 95% of the trans-fatty acids within the human diet. The consumption of fatty acids from dietary sources has both positive and negative implications for health. Consumption of trans-fatty acids have a direct correlation with various health problems including thrombogenesis which leads to increased risk for coronary heart and cardiovascular diseases, increased levels of low-density lipoproteins, and decreased levels of high-density lipoproteins. Significant consumption of trans-fatty acids also leads to an increased risk of diabetes from a rise in blood insulin levels when these fats are consumed. In 2011 the United States government banned the sale of food with more than 2 grams of trans-fatty acid per 100 grams of oil or fat. In the US, food with trans-fatty acid levels of less than 0.5 grams per serving can be labeled as “Trans Fat Free”, but critics of the this plan have expressed concern that the 0.5 gram per serving threshold is too high to refer to a food as free of trans fat. A person eating many servings of a product or eating multiple products over the course of the day, may still consume significant amounts of trans-fatty acids. In contrast to trans-fatty acids, the consumption of cis-fatty acids are often associated with positive health benefits.

Although partially hydrogenated vegetable oils account for 95% of the trans-fatty acids that are consumed each year, virgin vegetable oils do not possess any trans-fatty acids prior to hydrogenation. For example, over 35 million tons of soybean oil are produced each year, and it has a composition of 10% palmitic acid, 4% stearic acid, 18% oleic acid, 55% linoleic acid, and 13% linolenic acid (palmitic oil is a 16 carbon saturated fatty acid, see FIG. 1 for the structures of certain fatty acids). One major limitation with soybean oil as a food source is that it has a significant fraction of polyunsaturated fats that are problematic for applications in food and animal feed. Polyunsaturated fatty acids are prone to oxidation which leads to rancidity and off flavors. Hydrogenation is the most common method to lower the levels of polyunsaturated fatty acids (over 10 million tons of partially hydrogenated soybean oil is produced each year, primarily for human consumption), but this process leads to the formation of significant amounts of trans-fatty acids.

In addition to their uses in food, vegetable oils are the most important renewable feedstock for the chemical industry and have grown by 5% a year since 2000. Despite the large scale production of vegetable oils and the fact that they are a critical biorenewable source of starting materials, both the oils and their fatty acids are used only in small quantities in industrial applications. Over 96% of vegetable oils are “burned” by humans or animals after being consumed or in engines when used as biodiesel.

A critical reason for the lack of applications of fatty acids as a starting material for industrial applications is that it is not possible to separate a mixture of fatty acids into individual components on a scale of millions of tons per year. For example, fatty acids isolated from vegetable oils are a mixture of five or more different fatty acids with different reactivities and that will yield different products after a reaction. Thus, when a mixture of five fatty acids derived from soybean oil are used as starting materials in an industrial process, many different products are obtained. The challenge of utilizing a mixture of fatty acids as starting materials limits their broader transformations into more valuable commercial products.

Methods to purify fatty acids include selective precipitation, liquid chromatography or selective hydrolysis of trans-fatty acids from glycerol. Although each method has found applications on small batches of fatty acids, none of them can separate complex mixtures of fatty acids into individual components on the scale of millions of tons per year that is required for widespread applications.

Accordingly, there is a need for better methods to separate mixtures of two or more different fatty acids. In particular, there is a need for better methods to separate a mixture of two or more different fatty acids to provide an enriched mixture of two or more fatty acids or to provide an individual fatty acid that has been enriched.

### SUMMARY OF THE INVENTION

Membranes are commonly used in industry to remove impurities from a mixture of molecules. Separations using membranes are a preferred method for large industrial applications because it is one of the simplest and least energy intensive methods of purification. Membranes have been used to remove impurities (i.e. proteins and glycerols) from fatty acids, however, they have not been used to separate a mixture of two or more fatty acids to provide an enriched mixture of fatty acids or individual fatty acids. One reason it is difficult to separate fatty acids using membranes is that fatty acids are similar in size and polarity. Although cis and trans double bonds confer differences in overall shape to fatty acids, the ease of rotation about the numerous carbon-carbon sigma bonds leads to a large number of energetically assessable conformations for each fatty acid which increases the complexity of separating them with membranes.

Applicant has discovered that when fatty acids are associated with counterions (e.g. a fatty acid salt) their critical size is increased and the resulting fatty acid salts can be separated via a membrane. In one example a mixture of fatty acid salts were separated wherein cis-fatty acid salts were selectively retained by polydicyclopentadiene (PDCPD) membranes and the saturated and trans-fatty acid salts readily permeated the membranes and were not retained. This represents a significant new method to isolate fatty acids including individual fatty acids and mixtures of two or more fatty acids.

Accordingly, in one embodiment the invention provides a method for separating a mixture of two or more fatty acids associated with counterions comprising contacting a membrane with a first mixture comprising two or more different fatty acids associated with counterions, so that the mixture is fractionated into a permeate comprising one or more different fatty acids associated with counterions and a retentate comprising one or more different fatty acids associated with counterions, wherein at least one of the permeate or retentate is enriched in one or more different fatty acids associated with counterions.

### BRIEF DESCRIPTION OF THE FIGURES

FIG. 1 illustrates the structure of certain fatty acids.

FIG. 2 illustrates the retention of molecules based on molecular weight and critical area. A retention of 100% indicated that the molecule did not permeate the membrane at any level and a retention of 0% indicated that the molecule readily permeated the membrane and was not retained. a) The plot of retention versus molecular weight for 35 samples is shown. b) The plot of retention versus critical area is shown for the molecules in part a).

FIG. 3 shows a schematic of the apparatus that was used in most of the experiments described herein. Molecules A and B were initially added to the solvent upstream of the membrane and only molecule A permeated to the downstream solvent.

FIG. 4 illustrates the values for Sd/Su at 24, 48, and 72 h for stearic, linoleic, vaccenic, petroselinic, and linolenic acid. The lines are meant for ease of viewing the data and do not represent a fit to any equation.

FIG. 5 illustrates energy-minimized space filling models for each fatty acid and fatty acid salt with triisobutylamine. One image shows the fatty acid to emphasize any curvature. The other image shows a view of the critical area of each fatty acid salt with triisobutylamine. The images for a) elaidic acid, b) stearic acid, c) oleic acid, d) linoleic acid, e) linolenic acid, f) vaccenic acid, and g) petroselinic acid are shown.

### DETAILED DESCRIPTION

Alkyl as used herein in includes straight and branched saturated hydrocarbon chains. Alkenyl as used herein includes straight and branched hydrocarbon chains that comprise one or more carbon-carbon double bonds.

Alkynyl as used herein includes straight and branched hydrocarbon chains that comprise one or more carbon-carbon triple bonds.

Cycloalkyl such as a (C₃-C₈)cycloalkyl or (C₃-C₇)cycloalkyl) as used herein refers to a saturated or partially unsaturated cyclic hydrocarbon.

Optionally substituted alkyl, optionally substituted alkenyl and optionally substituted alkynyl include alkyl, alkenyl and alkynyl groups optionally substituted with one or more (e.g. 1, 2, 3, 4, 5, or more) groups independently selected from, oxo (═O), halo, —OR^{a}, —NR^{a}₂ and —NR^{a}₃ wherein each R^{a} is independently selected from H, (C₁-C₆)alkyl, (C₂-C₆)alkenyl, (C₂-C₆)alkynyl and (C₃-C₇)cycloalkyl).

Optionally substituted cycloalkyl groups include cycloalkyl groups optionally substituted with one or more (e.g. 1, 2, 3, 4, 5, or more) groups independently selected from (C₁-C₆)alkyl, (C₁-C₆)alkenyl, (C₁-C₆)alkynyl, oxo (═O), halo, —OR^{a}, —NR^{a}₂ and —NR^{a}₃ wherein each R^{a} is independently selected from H, (C₁-C₆)alkyl, (C₂-C₆)alkenyl, (C₂-C₆)alkynyl and (C₃-C₇)cycloalkyl).

Halo or halogen as used herein includes fluoro, chloro, bromo and iodo.

### Membranes

Membranes include semipermeable materials which can be used to separate components of a mixture into a permeate that passes through the membrane and a retentate that is rejected or retained by the membrane. One particular type of membrane is an organic solvent nanofiltration membrane. An organic solvent nanofiltration membrane is a membrane that is compatible with organic solvents and separates molecules in a specific size range. In one embodiment the organic nanofiltration membrane separates molecules with molecular weights from 50 to 1000 g mol⁻¹. Organic solvent nanofiltration membranes include but are not limited to those membranes based on polydicyclopentadiene, polyimide, polyaniline and polyacrylate which polymeric materials can be nanoparticulate. Examples include highly cross-linked polydicyclopentadiene (PDCPD), Duramem® (membrane), Puramem® (membrane), and Starmem® (membrane).

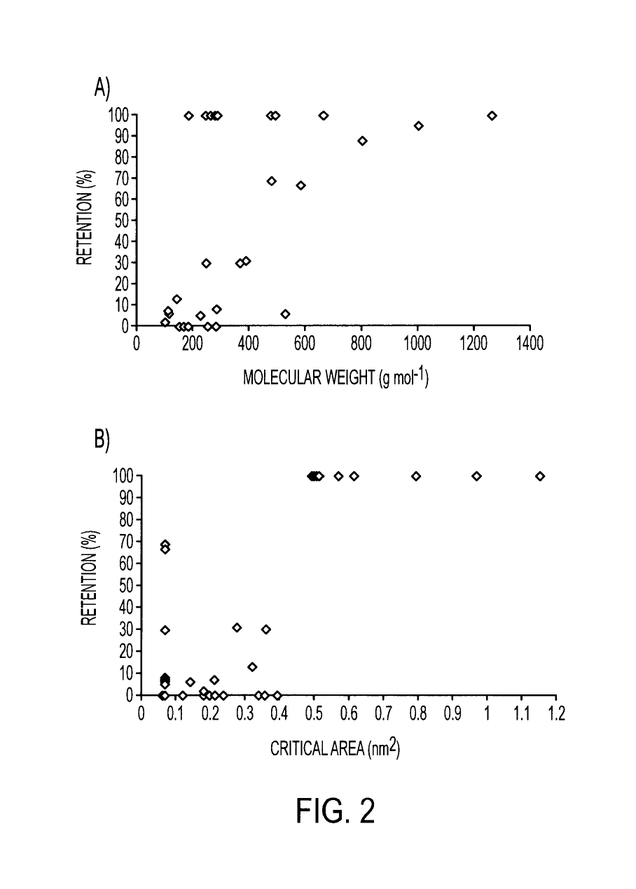

One particular membrane is highly cross-linked polydicyclopentadiene (PDCPD) (Long, T. R.; Gupta, A.; Miller I I, A. L.; Rethwisch, D. G.; Bowden, N. B. J. Mater. Chem. 2011, 21, 14265; Gupta, A.; Rethwisch, D. G.; Bowden, N. B. Chem. Commun. 2011, 46, 10236, and U.S. patent application Ser. No. 13/546,252, all of which references are hereby incorporated in their entirety). These membranes were fabricated by polymerizing 5,000 molar equivalents of dicyclopentadiene with one molar equivalent of the Grubbs first generation catalyst to yield solid, dense membranes. These membranes do not have well-defined pores such as zeolites; rather, when they are swollen in organic solvents, they possess openings between the polymer chains that small molecules may diffuse through. The distribution in size of the openings is polydisperse and on the nanometer to sub-nanometer size scale. The flux of a large number of molecules through PDCPD membranes were investigated and it was discovered that the membranes were highly selective to retain molecules with cross-sectional areas above 0.50 nm² as shown in FIG. 2, but molecules with cross-sectional areas below 0.38 nm² permeated the membranes (Gupta, A.; Rethwisch, D. G.; Bowden, N. B. Chem. Commun. 2011, 46, 10236.). The critical areas in FIG. 2 were defined as the smallest rectangular cross-sectional areas for each molecule in its lowest energy state. These values were measured in silico as reported in prior publications (Gupta, A.; Rethwisch, D. G.; Bowden, N. B. Chem. Commun. 2011, 46, 10236.) The molecules that were retained had values for their flux that were at least four to five orders of magnitude lower than those for molecules that did permeate. These membranes did a poor job of separating molecules based on their molecular weights as shown in FIG. 2a, but when the critical area for each molecule was plotted against retention a clear difference was observed. PDCPD membranes are a new type of size-selective membrane that separates organic molecules with molecular weights up to 600 g mol⁻¹ based on their cross-sectional areas.

As used herein the term “highly crosslinked” as applied to a polydicyclopentyldiene matrix includes matrices wherein the ratio of crosslinked double bonds to uncrosslinked double bonds is at least about 3:2. In one embodiment of the invention the ratio of crosslinked double bonds to uncrosslinked double bonds is about 2:3. In one embodiment of the invention the ratio of crosslinked double bonds to uncrosslinked double bonds is at least about 7:3. In another embodiment of the invention the ratio of crosslinked double bonds to uncrosslinked double bonds is at least about 4:1.

As used herein, the term “matrix” means a regular, irregular and/or random arrangement of polymer molecules such that on a macromolecular scale the arrangements of molecules may show repeating patterns, or may show series of patterns that sometimes repeat and sometimes display irregularities, or may show no pattern. On a scale such as would be obtained from TEM, SEM, X-Ray or FTNMR, the molecular arrangement may show a physical configuration in three dimensions like those of networks, meshes, arrays, frameworks, scaffoldings, three dimensional nets or three dimensional entanglements of molecules. The matrix may be non-self supporting. The matrix is in the form of a thin film with an average thickness from about 5 nm to about 100,000 nm. In usual practice, the matrix is grossly configured as an ultrathin film or sheet.

In one embodiment the invention provides a composite membrane comprising a highly crosslinked polydicyclopentyldiene matrix on a porous support backing material. The porous support backing material can comprise a polymeric material containing pore sizes which are of sufficient size to permit the passage of permeate therethrough. Examples of porous support backing materials which may be used to prepare composite membranes of the invention include polymers such as polysulfones, polycarbonates, microporous polypropylenes, polyamides, polyimines, polyphenylene ethers, and various halogenated polymers such as polyvinylidine fluoride.

Fatty Acids

The term “fatty acid” as used herein refers to an aliphatic carboxylic acid. The aliphatic group of the fatty acid is a hydrocarbon chain of about 4-28 carbons and can be straight or branched (e.g. a (C₅-C₂₉) fatty acid). Fatty acids include saturated fatty acids (e.g. fatty acids wherein the aliphatic group is saturated such as a (C₄-C₂₈)alkyl) and unsaturated fatty acids (e.g. fatty acids wherein the aliphatic group has at least one carbon-carbon double bond such as a (C₄-C₂₈)alkenyl). Unsaturated fatty acids include monounsaturated fatty acids (fatty acids wherein the aliphatic group has one carbon-carbon double) and polyunsaturated fatty acids (fatty acids wherein the aliphatic group has two or more carbon-carbon double bonds).

Fatty acids include but are not limited to oleic acid, linolenic acid, vaccenic acid, petroselinic acid, elaidic acid, palmitic acid, stearic acid, omega 3 fatty acids (e.g. linolenic acid, eicosapetnaenoic acid and docosahexaenoic acid), omega 6 fatty acids (e.g. linoleic acid and arachidonic acid) and omega 9 fatty acids.

The term “cis-fatty acid” refers to a an unsaturated fatty acid that has at least one cis carbon-carbon double bond in the aliphatic group (e.g. cis-(C₄-C₂₈)alkenylCO₂H). Examples of cis-fatty acids include but are not limited to oleic acid, linoleic acid, linolenic acid, vaccenic acid, petroselinic acid, eicosapetnaenoic acid, docosahexaenoic acid and arachidonic acid.

The term “trans-fatty acid” refers to an unsaturated fatty acid that has at least one trans carbon-carbon double bond and no cis carbon-carbon double bonds in the aliphatic group (e.g. trans-(C₄-C₂₈)alkenylCO₂H). Examples of trans-fatty acids include but are not limited to elaidic acid.

### Counterions

The fatty acids discussed herein form complexes with counterions. These complexes comprise the fatty acid and a counterion wherein the counterion is associated with the carboxyl portion of the fatty acid. As used herein the term “complex” includes any structure comprising a fatty acid and a counterion; such complexes include salts. As used herein the term “associated” includes any interaction (e.g. ionic, electrostatic, bonding, etc.) between the fatty acid and the counterion. In one embodiment each fatty acid is associated with one counterion. Counterions as used herein include molecules that modify the critical area of the fatty acid in manner such that fatty acids can be separated by membrane. In one embodiment the counterion is considered to be positively charged and is associated with the carboxylate anion of the fatty acid (such complexes includes fatty acid salts). In one embodiment the counterion is a positively charged amine. In one embodiment the protonated amine counterion can be derived by the interaction of an amine with the acidic hydrogen of the fatty acid.

Amines as used herein include mono, di, tri and tetrasubstituted amines wherein the substituents are independently selected from optionally substituted alkyl, optionally substituted alkenyl, optionally substituted alkynyl and optionally substituted cycloalkyl groups Amines also include cyclic amines. The term “cyclic amine” refers to a cycloalkyl wherein one or more (e.g. 1, 2 or 3) of the carbon atoms of the cycloalkyl have be replaced with one or more nitrogen atoms and wherein one or more of the carbon atoms (e.g. 1 or 2) have been optionally replaced with a heteroatom selected from oxygen and sulfur. Such cyclic amines include but are not limited to azetidinyl, pyrrolidinyl, morpholinyl, thiomorpholinyl, piperazinyl, homopiperazinyl and piperidinyl. The term “optionally substituted cyclic amine” includes cyclic amines that are optionally substituted with one or more (e.g. 1, 2, 3, 4, 5, or more) groups independently selected from (C₁-C₆)alkyl, (C₂-C₆)alkenyl, (C₂-C₆)alkynyl, oxo (═O), halo, —OR^{a}, —NR^{a}₂ and —NR^{a}₃ wherein each R^{a} is independently selected from H, (C₁-C₆)alkyl, (C₂-C₆)alkenyl, (C₂-C₆)alkynyl and (C₃-C₇)cycloalkyl).

Positively charged amines include those amines described above that are positively charged and included tetrasubstituted amines including tetrasubstituted cyclic amines and protonated amines (e.g. amines (including cyclic amines) that are positively charged and have at least one hydrogen on the amine nitrogen).

### Solvents

Any suitable organic solvent can be used with the fatty acids in the separations described herein. For example, suitable solvents may include protic and aprotic organic solvents (e.g. methanol, benzene, toluene, methylene chloride, chloroform, carbontetrachloride, tetrahydrofuran, pentane, hexanes, dimethylformamide or acetonitrile) or mixtures thereof.

### Specific Embodiments of the Invention

It is to be understood that the following embodiments of the invention can be combined with one or additional embodiments of the invention as described herein.

In one embodiment the invention provides a method wherein the permeate is enriched in one or more different fatty acids associated with counterions.

In one embodiment the invention provides a method wherein the retentate is enriched in one or more different fatty acids associated with counterions.

In one embodiment the invention provides a method wherein the permeate is enriched in one or more different fatty acids associated with counterions and the retentate is enriched in one or more different fatty acids associated with counterions.

In one embodiment the invention provides a method wherein the membrane is an organic solvent nanofiltration membrane.

In one embodiment the invention provides a method wherein the organic solvent nanofiltration membrane comprises polydicyclopentadiene, polyimide, polyaniline or polyacrylate.

In one embodiment the invention provides a method wherein the organic solvent nanofiltration membrane comprises polydicyclopentadiene.

In one embodiment the invention provides a method wherein the organic solvent nanofiltration membrane comprises a highly crosslinked polydicyclopentadiene matrix.

In one embodiment the invention provides a method wherein the ratio of crosslinked double bonds to uncrosslinked double bonds in the highly cross-linked polydicyclopentadiene matrix is at least about 2:3.

In one embodiment the invention provides a method wherein the ratio of crosslinked double bonds to uncrosslinked double bonds in the highly cross-linked polydicyclopentadiene matrix is at least about 3:2.

In one embodiment the invention provides a method wherein the ratio of crosslinked double bonds to uncrosslinked double bonds in the highly cross-linked polydicyclopentadiene matrix is at least about 4:1.

In one embodiment the invention provides a method wherein the membrane is a part of an assembly that comprises two or more membranes.

In one embodiment the invention provides a method wherein membrane is part of a spiral wound module.

In one embodiment the invention provides a method wherein the membrane separates molecules based on their cross-sectional areas.

In one embodiment the invention provides a method wherein each fatty acid of the first mixture, permeate and retentate is associated with one counterion and wherein the counterions are identical.

In one embodiment the invention provides a method wherein the counterion has a critical area that allows one or more different fatty acids associated with the counterion of the first mixture to permeate the membrane at a higher rate than one or more different fatty acids associated with the counterion of the first mixture so that the permeate is enriched in one or more different fatty acids associated with the counterion.

In one embodiment the invention provides a method wherein the counterion has a critical area that impedes the permeation of one or more different fatty acids associated with the counterion of the first mixture through the membrane so that the retentate is enriched in one or more fatty acids associated with the counterion.

In one embodiment the invention provides a method wherein the counterion has a critical area that allows one or more fatty acids associated with the counterion of the first mixture to permeate the membrane so that the permeate is enriched in one or more fatty acids associated with the counterion.

In one embodiment the invention provides a method wherein the counterion has a critical area that prevents one or more fatty acids associated with the counterion of the first mixture to permeate the membrane so that the retentate is enriched in one or more fatty acids associated with the counterion.

In one embodiment the invention provides a method wherein the counterion has a critical area of about 0.38 nm².

In one embodiment the invention provides a method wherein the counterion has a critical area of 0.38±0.08 nm².

In one embodiment the invention provides a method wherein the counterion has a critical area of 0.38±0.04 nm².

In one embodiment the invention provides a method wherein the counterion has a critical area of 0.38±0.02 nm².

In one embodiment the invention provides a method wherein counterion is a positively charged amine.

In one embodiment a positively charged amine is:
(a) ⁺NR₄ wherein each R is independently hydrogen, optionally substituted (C₁-C₈)alkyl, optionally substituted (C₂-C₈)alkenyl optionally substituted, (C₂-C₈)alkynyl or optionally substituted (C₃-C₈)cycloalkyl or
(b) an optionally substituted cyclic amine wherein at least one of the nitrogen atoms which comprise the cyclic amine is positively charged.

In one embodiment a positively charged amine is ⁺NR₄ wherein each R is independently hydrogen, optionally substituted (C₁-C₈)alkyl, optionally substituted (C₂-C₈)alkenyl, optionally substituted (C₂-C₈)alkynyl or optionally substituted (C₃-C₈)cycloalkyl.

In one embodiment a positively charged amine is ⁺NR₄ wherein each R is independently hydrogen, (C₁-C₈)alkyl, (C₂-C₈)alkenyl, (C₂-C₈)alkynyl or (C₃-C₈)cycloalkyl.

In one embodiment a positively charged amine is ⁺NR₄ wherein each R is independently hydrogen or (C₁-C₈)alkyl.

In one embodiment a positively charged amine (e.g. protonated amine) is ⁺NHR₃ wherein each R is independently optionally substituted (C₁-C₈)alkyl, optionally substituted (C₂-C₈)alkenyl, optionally substituted (C₂-C₈)alkynyl or optionally substituted (C₃-C₈)cycloalkyl.

In one embodiment a positively charged amine (e.g. protonated amine) is ⁺NHR₃ wherein each R is independently (C₁-C₈)alkyl, (C₂-C₈)alkenyl, (C₂-C₈)alkynyl or (C₃-C₈)cycloalkyl.

In one embodiment a positively charged amine (e.g. protonated amine) is ⁺NHR₃ wherein each R is independently (C₁-C₈)alkyl.

In one embodiment a positively charged amine (e.g. protonated amine) is ⁺NH₂R₂ wherein each R is independently (optionally substituted (C₁-C₈)alkyl, optionally substituted (C₂-C₈)alkenyl, optionally substituted (C₂-C₈)alkynyl or optionally substituted (C₃-C₈)cycloalkyl.

In one embodiment a positively charged amine (e.g. protonated amine) is ⁺NH₃R wherein each R is independently optionally substituted (C₁-C₈)alkyl, optionally substituted (C₂-C₈)alkenyl, optionally substituted (C₂-C₈)alkynyl or optionally substituted (C₃-C₈)cycloalkyl.

In one embodiment a positively charged amine is ⁺NR₄ wherein each R is independently optionally substituted (C₁-C₈)alkyl, optionally substituted (C₂-C₈)alkenyl, optionally substituted (C₂-C₈)alkynyl or optionally substituted (C₃-C₈)cycloalkyl.

In one embodiment the invention provides a method wherein counterion is a protonated amine.

In one embodiment the invention provides a method wherein the counterion is a tetraalkylammonium, trialkylammonium, dialkylammonium or monalkylammonium.

In one embodiment the invention provides a method wherein the counterion is a tetralkylammonium, trialkylammonium or dialkylammonium.

In one embodiment the invention provides a method wherein the counterion is a trialkylammonium.

In one embodiment the invention provides a method wherein the counterion is triisobutylammonium.

In one embodiment the invention provides a method wherein the first mixture comprises:

a) at least one cis-fatty acid associated with a counterion and

b) at least one trans-fatty acid associated with a counterion or at least one saturated fatty acid associated with a counterion.

In one embodiment the invention provides a method wherein the first mixture comprises at least one cis-fatty acid associated with a counterion and at least one saturated fatty acid associated with a counterion.

In one embodiment the invention provides a method wherein the first mixture comprises two or more different cis-fatty acids associated with counterions.

In one embodiment the invention provides a method wherein the permeate is enriched in at least one fatty acid associated with a counterion wherein the fatty acid associated with a counterion has critical area of less than or equal to about 0.42 nm².

In one embodiment the invention provides a method wherein the permeate is enriched in at least one fatty acid associated with a counterion wherein the fatty acid associated with a counterion has critical area of less than or equal to about 0.38 nm².

In one embodiment the invention provides a method wherein the permeate is enriched in at least one fatty acid associated with a counterion wherein the fatty acid has critical area of less than or equal to about 0.12 nm².

In one embodiment the invention provides a method wherein the permeate is enriched in at least one fatty acid associated with a counterion wherein the fatty acid has critical area of less than or equal to about 0.07 nm².

In one embodiment the invention provides a method wherein the permeate is enriched in at least one fatty acid associated with a counterion wherein the fatty acid associated with a counterion has critical area of less than or equal to about 0.65 nm².

In one embodiment the invention provides a method wherein the permeate is enriched in at least one fatty acid associated with a counterion wherein the fatty acid associated with a counterion has critical area of less than or equal to about 0.59 nm².

In one embodiment the invention provides a method wherein the permeate is enriched in at least one fatty acid associated with a counterion wherein the fatty acid has critical area of less than or equal to about 0.23 nm².

In one embodiment the invention provides a method wherein the permeate is enriched in at least one fatty acid associated with a counterion wherein the fatty acid has critical area of less than or equal to about 0.21 nm².

In one embodiment the invention provides a method wherein the permeate is enriched in at least one trans-fatty acid associated with a counterion or one saturated fatty acid associated with a counterion.

In one embodiment the invention provides a method wherein the permeate is enriched in at least one saturated fatty acid associated with a counterion.

In one embodiment the invention provides a method wherein the permeate is enriched in at least one cis-fatty acid associated with a counterion.

In one embodiment the invention provides a method wherein the retentate is enriched in at least one fatty acid associated with a counterion wherein the fatty acid associated with a counterion has critical area of greater than or equal to about 0.53 nm².

In one embodiment the invention provides a method wherein the retentate is enriched in at least one fatty acid associated with a counterion wherein the fatty acid associated with a counterion has critical area of greater than or equal to about 0.59 nm².

In one embodiment the invention provides a method wherein the retentate is enriched in at least one fatty acid associated with a counterion wherein the fatty acid associated with a counterion has critical area of greater than or equal to about 0.83 nm².

In one embodiment the invention provides a method wherein the retentate is enriched in at least one fatty acid associated with a counterion wherein the fatty acid has critical area of greater than or equal to about 0.19 nm².

In one embodiment the invention provides a method wherein the retentate is enriched in at least one fatty acid associated with a counterion wherein the fatty acid has critical area of greater than or equal to about 0.21 nm².

In one embodiment the invention provides a method wherein the retentate is enriched in at least one fatty acid associated with a counterion wherein the fatty acid has critical area of greater than or equal to about 0.29 nm².

In one embodiment the invention provides a method wherein the retentate is enriched in at least one cis-fatty acid associated with a counterion.

In one embodiment the invention provides a method wherein the first mixture comprises soybean oil wherein the fatty acid components of the soybean oil are associated with counterions.

In one embodiment the invention provides a method wherein the first mixture comprises palmitic acid, stearic acid, linolenic acid, oleic acid and linoleic acid each associated with a counterion.

In one embodiment the invention provides a method wherein the permeate is enriched in palmitic acid and stearic acid each associated with a counterion.

In one embodiment the invention provides a method wherein the retentate is enriched in linolenic acid, oleic acid and linoleic acid each associated with a counterion.

In one embodiment the invention provides a method wherein the first mixture comprises, linolenic acid, oleic acid and linoleic acid each associated with a counterion.

In one embodiment the invention provides a method wherein the permeate is enriched in oleic acid associated with a counterion.

In one embodiment the invention provides a method wherein the retentate is enriched in linolenic acid and linoleic acid each associated with a counterion.

In one embodiment the invention provides a method wherein the retentate is enriched in linolenic acid associated with a counterion.

In one embodiment the invention provides a method wherein the retentate is enriched in linoleic acid each associated with a counterion.

In one embodiment the invention provides a method wherein the permeate is removed one or more times during the separation.

In one embodiment the invention provides a method wherein the permeate is removed one or more times during the separation and replaced with a solvent.

In one embodiment the invention provides a method wherein the permeate is removed continuously.

In one embodiment the invention provides a method wherein the first mixture comprises a solvent.

In one embodiment the invention provides a method wherein the solvent comprises one or more protic or aprotic organic solvents.

In one embodiment the invention provides a method wherein the solvent comprises toluene, hexane, methanol, methylene chloride, tetrahydrofuran, dimethylformamide, chloroform, benzene or acetonitrile or mixtures thereof.

In one embodiment the invention provides a method wherein the solvent comprises toluene, hexane, methanol or methylene chloride or mixtures thereof.

In one embodiment the invention provides a method wherein the solvent comprises methanol and methylene chloride.

In one embodiment the invention provides a method wherein pressure is applied to the first mixture to increase the flux of the first mixture through the membrane.

In one embodiment the invention provides a mixture comprising two or more different fatty acids wherein the fatty acids are associated with a counterion, wherein the counterion has a critical area of 0.38 nm²±0.04 nm².

In one embodiment the counterion has a critical area of 0.38±0.02 nm².

In one embodiment the counterion has a critical area of about 0.38 nm².

In one embodiment the counterion is triisobutylammonium.

In one embodiment each fatty acid of the mixture is associated with one counterion and wherein the counterions are identical.

In one embodiment the invention provides a mixture which comprises:

a) at least one cis-fatty acid associated with a counterion and

b) at least one trans-fatty acid associated with a counterion or at least one saturated fatty acid associated with a counterion.

In one embodiment the mixture comprises two or more cis-fatty acids associated with counterions.

In one embodiment the mixture comprises palmitic acid, stearic acid, linolenic acid, oleic acid and linoleic acid each associated with a counterion.

In one embodiment the mixture comprises linolenic acid, oleic acid and linoleic acid each associated with a counterion.

The invention will now be illustrated by the following non-limiting Example.

### Example 1

### Separation of Fatty Acids

Materials.

Dicyclopentadiene, elaidic acid, oleic acid, stearic acid, linoleic acid, linolenic acid, vaccenic acid, petroselinic acid, triethylamine, tripropylamine, triisobutylamine, tributylamine, p-nitrobenzaldehyde, and solvents were purchased at their highest purity from Aldrich and Acros and used as received.

Characterization.

¹H NMR spectra were acquired using a Bruker DPX-500 at 500 MHz and referenced to TMS.

Fabrication of PDCPD Membranes.

A 20 mg mL⁻¹ solution of Grubbs first generation catalyst (benzylidene-bis(tricyclohexylphosphine)dichlororuthenium, bis(tricyclohexylphosphine)-benzylidine ruthenium(IV)dichloride) was made using 1,2-dichloroethane. A sample of this solution (0.72 mL, 6.0×10⁻³ mmol of catalyst) was added to 12 mL of dicyclopentadiene and heated to 40° C. Heat was used to keep dicyclopentadiene (melting point 33° C.) a liquid. This solution was immediately placed between two glass slides with 100 μm thick paper as spacers along the edges. The sample was heated to 50° C. for 2 h and then removed from the glass slides. All PDCPD membranes used as described herein were fabricated according to this method.

Permeation of Oleic Acid and Elaidic Acid with Different Amines (Results Shown in Table 1).

A PDCPD membrane was added to the apparatus to study permeation. CH₂Cl₂:MeOH (v/v, 75:25, 25 mL) was added to the downstream side of the membrane and 25 mL of the same solvent was added to the upstream side of the membrane with 0.426 mmol of oleic acid, 0.426 mmol of elaidic acid, 0.852 mmol of amine and 0.426 mmol of p-nitrobenzaldehyde as an internal standard. Solvent on both sides of the membrane was stirred continuously at room temperature. At 24, 48, and 72 h a 1 mL aliquot of solvent was removed from solvent on both sides of the membrane. The aliquots were used to determine the concentration and the absolute amounts of the oleic acid salt, elaidic acid salt, and p-nitrobenzaldehyde by ¹H NMR spectroscopy. The S_{d}/Sᵤ values were found by the addition of known amounts of toluene to each aliquot as an internal standard.

Permeation of Saturated Cis-Fatty Acid Salts with Triisobutylamine Through PDCPD (Results Shown in Table 2).

A PDCPD membrane was added to the apparatus to study permeation. CH₂Cl₂:MeOH (v/v, 75:25, 25 mL) was added to the downstream side of the membrane and 25 mL of the same solvent was added to the upstream side of the membrane with 0.426 mmol of stearic acid, 0.426 mmol of unsaturated acid, 0.852 mmol of triisobutylamine, and 0.426 mmol p-nitrobenzaldehyde as an internal standard. Both sides of the membrane were stirred continuously at room temperature. At 24, 48, and 72 h a 1 mL aliquot of solvent was removed from both sides of the membrane. The aliquots were used to determine the concentration and the absolute amounts of the stearic acid salt, unsaturated acid salt, and p-nitrobenzaldehyde by ¹H NMR spectroscopy. The S_{d}/Sᵤ values were found by the addition of known amounts of toluene as an internal standard to each aliquot.

Permeation of Stearic and Oleic Acid as Triisobutylamine Salts Through PDCPD in Different Solvents (Results Shown in Table 3).

A PDCPD membrane was added to the apparatus to study permeation. Toluene or chloroform (25 mL) was added to the downstream side of the membrane and 25 mL of the same solvent was added to the upstream side of the membrane with 0.426 mmol of stearic acid, 0.426 mmol of oleic acid, 0.852 mmol of triisobutylamine, and 0.426 mmol p-nitrobenzaldehyde as an internal standard. Solvent on both sides of the membrane were stirred continuously at room temperature. At 24, 48, and 72 h a 1 mL aliquot of solvent was removed from both sides of the membrane. The aliquots were used to determine the concentration and the absolute amounts of the stearic acid salt, oleic acid salt, and p-nitrobenzaldehyde by ¹H NMR spectroscopy. The S_{d}/Sᵤ values were found by the addition of known amounts of tetraethylene glycol as an internal standard to each aliquot. Partition Coefficients of Molecules in PDCPD (Results Shown in Table 4).

A PDCPD slab was cut into small rectangular pieces. A typical value for the dimension of the slab was 2.5 cm×0.9 cm×0.3 cm, and the weight was approximately 0.800 g. A fatty acid (0.213 mmol) and triisobutylamine (0.213 mmol) was dissolved in 12.5 mL of CH₂Cl₂:MeOH (v/v, 75:25) solution. The weight of the PDCPD slab was measured, and it was immersed in the solution. The solution was stirred for 96 h. After 96 h, the PDCPD slab was pulled out of the solution and solvent was removed in vacuo. The weight of the slab was measured. The amount of molecule that partitioned into the slab was calculated based on the difference in weight of the slab before and after being swollen. The volume of the solvent was measured prior to removing it in vacuo. An aliquot of the residue was added to a NMR tube. The absolute amount of the fatty acid in the solvent was determined by ¹H NMR spectroscopy by the addition of known amounts of tetraethylene glycol. The partition coefficient of the molecule was calculated by dividing the concentration of the molecule in PDCPD by the concentration of the molecule in the solvent.

Critical Areas of Fatty Acids (Results Shown in Table 5).

The software used for these calculations was Spartan '08 V1.2.0. The model for each molecule was drawn in the software using a space filling model. The energy was minimized for each molecule using a semi-empirical AM1 to find the conformation with the lowest energy. Each molecule was thoroughly visualized to see which conformation had the lowest cross-sectional area. This method has been previously described (Long, T. R.; Gupta, A.; Miller I I, A. L.; Rethwisch, D. G.; Bowden, N. B. J. Mater. Chem. 2011, 21, 14265; Gupta, A.; Rethwisch, D. G.; Bowden, N. B. Chem. Commun. 2011, 46, 10236).

Separation of a Mixture of Four Fatty Acids Using Multiple Extractions.

A PDCPD membrane was added to the apparatus to study permeation. CH₂Cl₂ (25 mL) was added to the downstream side of the membrane and 25 mL of the same solvent was added to the upstream side of the membrane with 0.426 mmol of stearic acid, 0.426 mmol of oleic acid, 0.426 mmol of linoleic acid, 0.426 mmol of linolenic acid, and 1.704 mmol of triisobutylamine. Solvent on both sides of the membrane were stirred continuously at room temperature. After 24, 48, and 72 h the downstream solvent was removed and replaced with fresh 25 mL CH₂Cl₂. After 96 h, the downstream solvent was combined with the previous solvent removed from the downstream side of the membrane. After 96 h, the upstream solvent was removed and replaced with 25 mL of CH₂Cl₂ and 1.278 mmol of triethylamine to extract any fatty acid retained in the membrane. After 45 h, the solvent was replaced with 25 mL of CH₂Cl₂ for a second recovery cycle. The downstream and upstream solvents were combined separately to determine the absolute amounts of stearic acid salt, oleic acid salt, linoleic acid salt, and linolenic acid salt by ¹H NMR spectroscopy. The absolute amounts of each fatty acid were found by the addition of known amounts of tetraethylene glycol to each aliquot.

Use of Pressure to Increase the Flux Through PDCPD Membranes (Results Shown in Table 6).

A PDCPD membrane was immersed in 30 mL of CH₂Cl₂:MeOH (v/v, 90:10, 75:25, or 60:40) for 15 min. After 15 min, the membrane was added to a metal vessel to study flux. CH₂Cl₂:MeOH at the same v/v ratio (100 ml) was added to the upstream side of the membrane with 0.426 mmol of stearic acid, 0.426 mmol of oleic acid, and 0.852 mmol of triisobutylamine. The valve on the downstream side was opened. The pressure was increased to 90 psi in 10 min. After an induction period of a few hours where no solution permeated to the downstream side, the solution was collected on the downstream side in 15-20 min. An aliquot of solvent was used to determine the absolute amounts of stearic acid and oleic acid salts by ¹H NMR spectroscopy. The absolute amounts of the salts were found by the addition of known amounts of tetraethylene glycol to the aliquot. The same experiment was repeated with mixtures of toluene:hexane (v/v, 40:60, 35:65, or 30:70).

### Results and Discussion

Choice of Fatty Acids and how the Experiments were Completed.

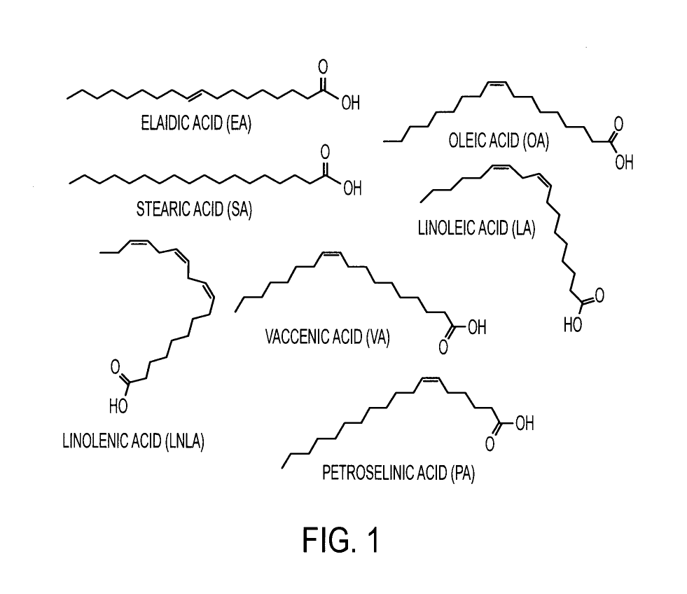

The fatty acids shown in FIG. 1 were used in the examples herein. These fatty acids all possessed 18 carbons and represented some of the most common fatty acids found in vegetable oils. Linolenic acid and linoleic acid which are two essential fatty acids that are needed within the body but that humans are not able to synthesize are also included in the examples described herein.

PDCPD membranes were fabricated as described in the experimental section (Scheme 1). These membranes were highly cross-linked by the Grubbs catalyst. Dicyclopentadiene has two carbon-carbon pi bonds; one is highly strained (approximately 25 kcal/mole of ring strain) and other is less strained (approximately 7 kcal/mole of ring strain). The ring opening metathesis polymerization of the highly strained pi bond yielded polymer and the ring opening of the less strained ring yielded cross-links (Long, T. R.; Gupta, A.; Miller I I, A. L.; Rethwisch, D. G.; Bowden, N. B. J. Mater. Chem. 2011, 21, 14265; Gupta, A.; Rethwisch, D. G.; Bowden, N. B. Chem. Commun. 2011, 46, 10236; Amendt, M. A.; Chen, L.; Hillmyer, M. A. Macromolecules 2010, 43, 3924; Amendt, M. A.; Roerdink, M.; Moench, S.; Phillip, W. A.; Cussler, E. L.; Hillmyer, M. A. Aust. J. Chem. 2011, 64, 1074; Kovacic, S.; Krajnc, P.; Slugovc, C. Chem. Commun. 2010, 46, 7504; Lee, J. K.; Gould, G. L. J. Sol-Gel Sci. Technol. 2007, 44, 29; Ren, F.; Feldman, A. K.; Carnes, M.; Steigerwald, M.; and Nuckolls, C. Macromolecules 2007, 40, 8151; Rule, J. D.; Moore, J. S. Macromolecules 2002, 35, 7878). In prior work it was shown that 83% of the less strained pi bond was ring opened which led to a highly cross-linked matrix (Long, T. R.; Gupta, A.; Miller I I, A. L.; Rethwisch, D. G.; Bowden, N. B. J. Mater. Chem. 2011, 21, 14265; Gupta, A.; Rethwisch, D. G.; Bowden, N. B. Chem. Commun. 2011, 46, 10236). Scheme 1 shows the polymerization of dicyclopentadiene by the Grubbs first generation catalyst yielded a highly cross-linked solid polymeric slab.

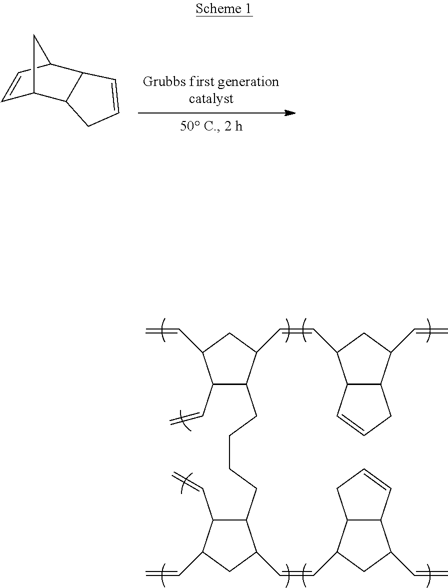

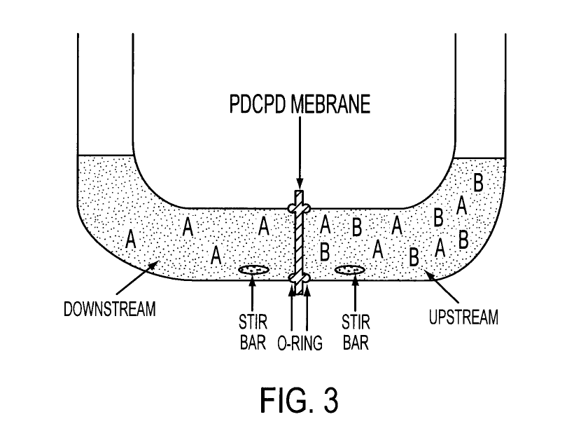

The experimental apparatus for studying the permeation of fatty acids is shown in FIG. 3. A 100 micron-thick PDCPD membrane was fabricated and placed between two glass reservoirs. Solvent was added to either side of the membrane. On one side of the membrane molecules that were to be studied were added with the solvent. This side was defined as the upstream side of the membrane. On the downstream side of the membrane only solvent was added. The molecules diffused from the upstream side to the downstream side of the membrane. The solvent on both sides of the membranes were constantly mixed using stir bars to ensure uniform concentrations on each side of the membrane. After a period of time, typically 24 h, well-defined aliquots of solvent from both sides of the membranes were removed and the solvent was evaporated. An internal standard of toluene or tetraethylene glycol was added prior to analysis by ¹H NMR spectroscopy to allow the absolution concentrations of each molecule downstream and upstream of the membranes to be measured.

Separation of Oleic Acid and Elaidic Acid.

The separation of oleic acid from elaidic acid was chosen as an initial test due to the similarities of these fatty acids. They possess identical molecular formulas and the double bond is located at the same position, but oleic acid is the cis isomer and elaidic acid is the trans isomer. A 75/25 (v/v) mixture of CH₂Cl₂/MeOH was used because all fatty acids dissolved in this solvent and reasonable flux values were obtained.

A mixture of oleic acid, elaidic acid, and p-nitrobenzaldehyde was added as an internal standard on the upstream side of the membrane. The ratio of the concentration of each molecule in the solvent on the downstream side (S_{d}) to the upstream side (Sᵤ) was measured every 24 h as described herein. The S_{d}/Sᵤ ratio was zero at the beginning of the experiment because the molecules were only added to the upstream side of the membrane. The S_{d}/Sᵤ ratio was equal to one when a molecule had diffused through the PDCPD membrane such that its concentration was the same on both sides of the membrane. Both oleic and elaidic acid readily permeated the PDCPD membrane at similar rates and were fully equilibrated after 72 h (entry 1 in Table 1). This result was expected based on the small cross-sectional areas of these fatty acids.

TABLE 1
Permeation of oleic acid and elaidic acid with
different amines through PDCPD membranes.

| | | Oleic acid (S_{d}/Sᵤ) | | | Elaidic acid S_{d}/Sᵤ) | | |
|---|---|---|---|---|---|---|---|
| Entry | Salt | 24 h | 48 h | 72 h | 24 h | 48 h | 72 h |
| 1 | No salt | 0.26 | 0.72 | 1.04 | 0.27 | 0.73 | 1.08 |
| 2 | triethylamine | 0.09 | 0.36 | 1.03 | 0.11 | 0.34 | 1.08 |
| 3 | tripropylamine | 0.01 | 0.09 | 0.17 | 0.13 | 0.58 | 1.08 |
| 4 | triisobutylamine | 0.017 | 0.05 | 0.07 | 0.20 | 0.68 | 1.01 |
| 5 | tributylamine | 0.0 | 0.0 | 0.0 | 0.0 | 0.0 | 0.0 |

A series of trisubstituted amines were added to the fatty acids to investigate their effect on the observed permeation. From previous studies, it was known that triethylamine, tripropylamine, and triisobutylamine readily permeated the membranes but that tributylamine did not permeate at any detectable level. Triisobutylamine and tributylamine are constitutional isomers; yet, their flux differed by at least four to five orders of magnitude. The difference in flux was due to the smaller, compact shape of triisobutylamine (critical area of 0.38 nm²) compared to tributylamine (critical area of 0.50 nm²).

An amine was added to solvent on the upstream side of the membrane with the fatty acids to form a noncovalent bond (Scheme 2). The fatty acid and the amine formed a salt pair by transfer of the hydrogen from the acid to the nitrogen. Scheme 2 illustrates the addition of triisobutylamine to form a stable salt with the fatty acids.

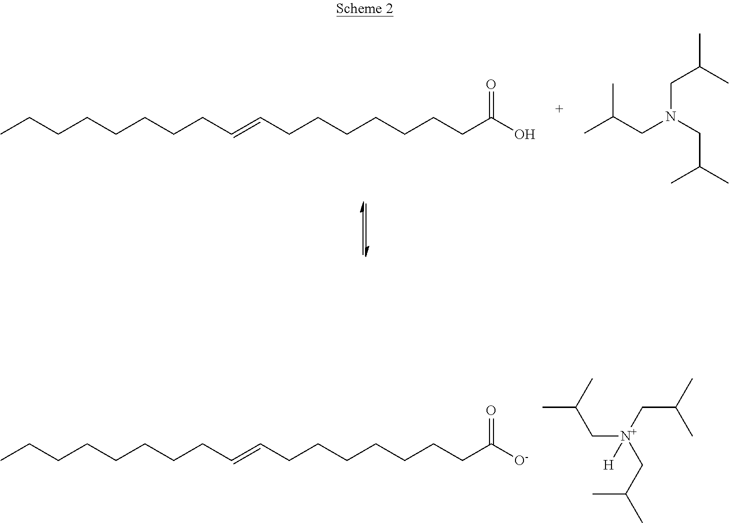 [SMILES: CCCCCCCC/C=C/CCCCCCCC(=O)O ; CC(C)CN(CC(C)C)CC(C)C ; CCCCCCCC/C=C/CCCCCCCC(=O)[O-] ; CC(C)C[NH+](CC(C)C)CC(C)C]

These salts were stable and persistent in a variety of organic solvents. The amines had compact shapes and larger cross-sectional areas than the fatty acids, so their addition increased the critical area of each fatty acid. It was hypothesized that the curvature of the cis-fatty acids would lead to a larger increase in their critical areas when compared to the saturated and trans-fatty acids. It was also hypothesized that the amine would increase the cross-sectional area of the fatty acids to reach the size range where PDCPD membranes were effective at separating molecules. The critical areas of each fatty acid and fatty acid salt are reported herein.

The results for the flux when a 1:1:2 molar ratio of oleic acid:elaidic acid:amine was added to the solvent on the upstream side of the membrane was shown in Table 1. The addition of triethylamine (critical area=0.18 nm²) had little impact on the flux of oleic and elaidic acid; both fatty acid salts equilibrated after 72 h. When tripropylamine (critical area=0.32 nm²) was added, the flux of oleic acid was slowed but elaidic acid equilibrated after 72 h. Better results were obtained when triisobutylamine (critical area=0.38 nm²) was used. The value for S_{d}/Sᵤ of oleic acid was only 0.07 after 72 h, but the elaidic acid was fully equilibrated. The use of tributylamine (critical area=0.50 nm²) kept both oleic and elaidic acid from permeating the membrane. Previous work demonstrated that tributylamine did not permeate these membranes at any detectable level, so it was expected that the salts would not permeate. This experiment with tributylamine demonstrated that the fatty acids coordinated strongly to the amines because if the fatty acids dissociated from tributylamine, they would have readily permeated the membranes.

Separation of Cis, Trans, and Saturated Fatty Acids.

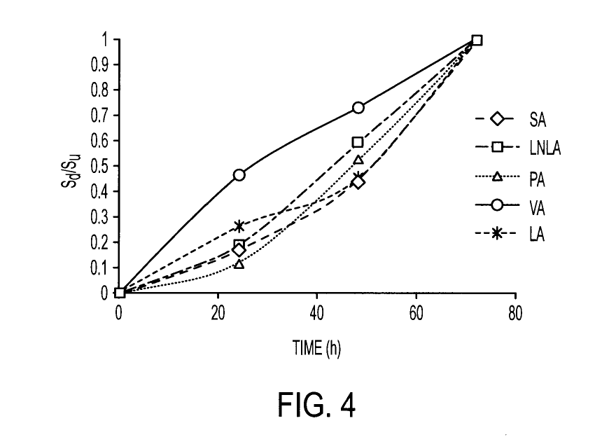

Five additional fatty acids were studied for their ability to permeate PDCPD membranes. Stearic, linoleic, vaccenic, petroselinic, and linolenic acid all readily permeated the membranes and fully equilibrated within 72 h (FIG. 4).

The results for the permeation of these five fatty acids were remarkably different when triisobutylamine was added to the solvent on the upstream side of the membrane (Table 2). When triisobutylamine was used, stearic acid readily permeated the membranes but the other four fatty acids had greatly reduced permeation. To ensure that the diminished permeation of the cis-fatty acids was due to their structure rather than another effect, the flux of each cis-fatty acid was studied in the presence of both stearic acid and p-nitrobenzaldehyde. The permeation of p-nitrobenzaldehyde was known from prior work, so it provided an internal standard for the physical properties of the membranes. In these experiments, both p-nitrobenzaldehyde and the stearic acid salt with triisobutylamine readily permeated the membranes. Thus, the diminished permeation of the cis-fatty acid salts was not due to the membranes, but rather it was due to their structures.

TABLE 2
Permeation of cis-fatty acid salts with
triisobutylamine through PDCPD membranes.

| | | Stearic acid (S_{d}/Sᵤ) | | | Unsaturated acid (S_{d}/Sᵤ) | | |
|---|---|---|---|---|---|---|---|
| Entry | ^{a}Fatty acids | 24 h | 48 h | 72 h | 24 h | 48 h | 72 h |
| 1 | Stearic acid and | 0.24 | 0.67 | 1.04 | 0.00 | 0.00 | 0.035 |
| | linoleic acid | | | | | | |
| 2 | Stearic acid and | 0.21 | 0.78 | 1.04 | 0.00 | 0.00 | 0.00 |
| | linolenic acid | | | | | | |
| 3 | Stearic acid and | 0.15 | 0.62 | 0.83 | 0.02 | 0.06 | 0.10 |
| | petroselinic acid | | | | | | |
| 4 | Stearic acid and | 0.40 | 0.65 | 1.00 | 0.03 | 0.07 | 0.11 |
| | vaccenic acid | | | | | | |

^{a}One molar equivalent of triisobutylamine to fatty acid was added to each experiment.

Two interesting sets of cis-fatty acids were studied in these experiments. Petroselinic, oleic, and vaccenic acid all possessed 18 carbons and one cis-olefin, but they differed in the location of the double bond (see FIG. 1). Petroselinic, oleic, and vaccenic acid had 11, 8, and 6 sp³ hybridized carbons after the double bond. The saturated ends of the fatty acids represented the “hooks” of the cis-fatty acids that led to their diminished flux through the membranes. All three of these fatty acids had similar flux through the membranes despite the difference in location of the cis bonds (entries 3 and 4 of Table 2 and entry 4 of Table 1). In a second set of cis-fatty acids, the number of cis bonds differed. Oleic, linoleic, and linolenic acids all possessed one cis-bond at the 9 carbon, but linoleic acid had a second cis-bond at the 12 carbon and linolenic acid had two additional cis-bonds at the 12 and 15 carbons. The number of cis bonds had a small effect on the flux of a fatty acid, but all of the cis-fatty acids were retained by the PDCPD membrane (entries 1 and 2 in Table 2 and entry 4 of Table 1).

In all prior experiments a mixture of 75/25 (v/v) of CH₂Cl₂/MeOH was used as the solvent. To investigate if the difference in flux for cis-fatty acids resulted in part from a choice of solvent, chloroform and toluene were studied (Table 3). In these experiments the permeation of stearic acid and oleic acid salts were examined to investigate how rapidly the stearic acid salt permeated and whether the oleic acid salt permeated. In both experiments the stearic acid salt readily permeated the membranes but the oleic acid salt did not permeate. The flux of the stearic acid salt was faster when toluene and chloroform were used as solvent than with the CH₂Cl₂/MeOH mixture. In the CH₂Cl₂/MeOH mixture the value for S_{d}/Sᵤ was 0.68 after 48 h (this value was the average of the four experiments shown in Table 2), but the value for S_{d}/Sᵤ after 48 h was 0.98 and 0.92 in toluene and chloroform respectively.

TABLE 3
Permeation of stearic acid and oleic acid as triisobutylamine
salts through PDCPD membranes in different solvents.

| | Stearic acid (S_{d}/Sᵤ) | | | Oleic acid (S_{d}/Sᵤ) | | |
|---|---|---|---|---|---|---|
| Solvent | 24 h | 48 h | 72 h | 24 h | 48 h | 72 h |
| Toluene | 0.32 | 0.98 | 1.00 | 0.03 | 0.06 | 0.05 |
| Chloroform | 0.34 | 0.92 | 1.00 | 0.03 | 0.06 | 0.07 |

Partitioning Coefficients for Fatty Acids and Fatty Acid Salts.

The equations that describe permeation can be complex, but the main concepts are straightforward (Balmer, T. E.; Schmid, H.; Stutz, R.; Delamarche, E.; Michel, B.; Spencer, N. D.; Wolf, H. Langmuir 2005, 21, 622; Banerjee, S.; Asrey, R.; Saxena, C.; Vyas, K.; Bhattacharya, A. J. Appl. Polym. Sci. 1997, 65, 1789; Crank, J. The mathematics of diffusion; Clarendon Press: Oxford, 1970; Du Pleiss, J.; Pugh, W. J.; Judefeind, A.; Hadgraft, J. Eur. J. Pharm. Sci. 2002, 15, 63; Philip, W. A.; Amendt, M.; O'Neill, B.; Chen, L.; Hillmyer, M. A.; Cussler, E. L. ACS Appl. Mater. Inter. 2009, 1, 472; Philip, W. A.; Martono, E.; Chen, L.; Hillmyer, M. A.; Cussler, E. L. J. Mem. Sci. 2009, 337, 39; Sarveiya, V.; Templeton, J. F.; Benson, H. A. E. Eur. J. Pharm. Sci. 2005, 26, 39; Shah, M. R.; Noble, R. D.; Clough, D. E. J. Mem. Sci. 2007, 287, 111 and Tamai, Y.; Tanaka, H.; Nakanishi, K. Macromolecules 1995, 28, 2544). For a molecule to permeate a membrane it must partition into the membrane, and it must have a nonzero rate of diffusion inside the membrane. The well-known equation P=DS describes this relationship (P is the permeability, D is the rate of diffusion, and S is the solubility of a molecule in the membrane). The partitioning coefficient, PC (unitless), is defined as the ratio of the concentration of a molecule in a membrane divided by its concentration in solvent when a system is at equilibrium. The partitioning coefficients for every fatty acid salt with triisobutylamine were investigated for their ability to permeate into PDCPD slabs as described in the supporting information.

The partitioning coefficients of oleic and elaidic acid in the absence of any amine were almost identical (entries 1 and 2 in Table 4). This result was expected based on the similarities of these fatty acids. Interestingly, the partitioning coefficients of all seven fatty acid with triisobutylamine were also nearly identical (entries 3-9 in Table 4). This result was due to the similarities in size and composition of the fatty acid salts and that the charged parts of the salts were encapsulated by the isobutyl groups and the hydrophobic tails of the fatty acids. The different in permeation of the cis-fatty acid salts compared to the saturated and trans-fatty acid salts was not their partitioning coefficients; rather, the differences were due to their rates of diffusion with the PDCPD matrix.

TABLE 4
Partitioning coefficients of fatty acids and fatty acid salts into PDCPD.

| Entry | Fatty acid | PC |
|---|---|---|
| ^{a}1 | Oleic acid | 1.07 |
| ^{a}2 | Elaidic acid | 0.997 |
| 3 | Elaidic acid salt | 0.999 |
| 4 | Oleic acid salt | 1.00 |
| 5 | Stearic acid salt | 1.00 |
| 6 | Linoleic acid salt | 0.999 |
| 7 | Linolenic acid salt | 1.00 |
| 8 | Petroselinic acid salt | 1.00 |
| 9 | Vaccenic acid salt | 1.00 |

^{a}The first two entries were measured as free acids without triisobutylamine. Entries 3-9 were measured with one molar equivalent of triisobutylamine present.

Measurement and Comparison of Critical Areas.

Differences in partitioning coefficients do not explain the differences in permeation of the fatty acid salts, so the differences in permeation must have been due to the differences in flux. In cross-linked polymer matrixes the diffusion, D, of a molecule depends exponentially on the energy of activation, Eₐ (kcal mol⁻¹) according to the equation D=Dₒexp(−Eₐ/RT) (Crank, J. The mathematics of diffusion; Clarendon Press: Oxford, 1970). Molecules that are much smaller than the pores in a matrix can diffuse rapidly because the polymer matrix does not have to rearrange to allow them to diffuse. Molecules that are on the same size as the pores or larger than the pores diffuse slowly because the polymer matrix must deform and the value for Eₐ is large. In practice, the rate of diffusion in cross-linked polymers has been shown to be heavily dependent on the cross-sectional areas of molecules. For instance, in 1982 Berens and Hopfenberg plotted the log of diffusion versus the square of diameter for 18 molecules that permeated poly(vinyl chloride), polystyrene, and polymethymethacrylate (Berens, A. R.; Hopfenberg, H. B. J. Mem. Sci. 1982, 13, 283). The diffusion of He (diameter squared=6.66×10⁻² nm²) was ten orders of magnitude faster than the diffusion of neopentane (diameter squared=3.36×10⁻¹ nm²). PDCPD was a highly cross-linked polymer matrix and the rate of diffusion of molecule was expected to depend on their critical areas. In prior work it was shown that molecules above a critical area of 0.50 nm² did not permeate PDCPD membranes but molecules with cross-sectional areas below 0.38 nm² did permeate.

One challenge in the field of size-selective membranes is defining the critical area of a molecule. This is usually not attempted; rather, membranes are described as possessing a “molecular weight cutoff” that is used to determine whether a new molecule will permeate (Fierro, D.; Boschetti-de-Fierro, A.; Abetz, V. J. Membr. Sci. 2012, 413-414, 91; Fritsch, D.; Merten, P.; Heinrich, K.; Lazar, M.; Priske, M. J. Membr. Sci. 2012, 401-402, 222; Rundquist, E. M.; Pink, C. J.; Livingston, A. G. Green Chem. 2012, 14, 2197; Sereewatthanawut, I.; Lim, F. W.; Bhole, Y. S.; Ormerod, D.; Horvath, A.; Boam, A. T.; Livingston, A. G. Org. Process Res. Dev. 2010, 14, 600; So, S.; Peeva, L. G.; Tate, E. W.; Leatherbarrow, R. J.; Livingston, A. G. Org. Process Res. Dev. 2010, 14, 1313; Szekely, G.; Bandana, J.; Heggie, W.; Sellergren, B.; Ferreira, F. C. J. Membr. Sci. 2011, 381, 21; and van, d. G. P.; Barnard, A.; Cronje, J.-P.; de, V. D.; Marx, S.; Vosloo, H. C. M. J. Membr. Sci. 2010, 353, 70). The molecular weight cutoff is used although it is not meant to be a good predictor of what will permeate. It is well understood that molecular weight does not have a strong correlation with cross-sectional area. Rather, a molecular weight cutoff provides a simple, unambiguous method to suggest which molecules may permeate a membrane. The molecular weight of a molecule can be determined within minutes, but the cross-sectional area is much harder to determine and dependent on the method used.

The critical areas for the molecules in this study were found using Spartan '08 V 1.2.0. The free fatty acids were constructed and their energies were minimized in Spartan. Not surprising, the fatty acids were in the all-trans conformations. The fatty acids were rotated until the smallest rectangular cross-sectional area was found, and this value was labeled the critical area and reported in Table 5. The critical area was measured because this area was the smallest size for the pore that each molecule may diffuse through. The procedure to find the critical areas for the fatty acids salts was similar. The energy of triisobutylamine was first minimized such that it could be docked in the same conformation with each fatty acid. Next, the energy of the fatty acid with the amine was minimized. The critical areas of the salts were found as described before.

TABLE 5
Critical areas of fatty acids.

| | | Critical area |
|---|---|---|
| | Critical area of | of fatty acid |
| | free fatty acid | salt |
| Molecule | (nm²) | (nm²) |
| Elaidic acid | 0.12 | 0.38 |
| Stearic acid | 0.067 | 0.38 |
| Oleic acid | 0.21 | 0.59 |
| Linoleic acid | 0.34 | 0.97 |
| Linolenic acid | 0.36 | 0.94 |
| Petroselinic acid | 0.20 | 1.27 |
| Vaccenic acid | 0.24 | 0.47 |

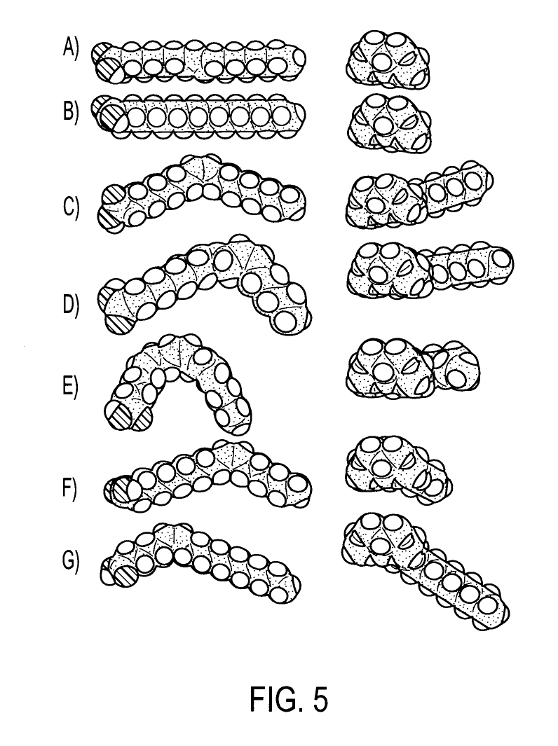

In FIG. 5 the seven fatty acid and fatty acids salts are shown in their energy minimized structures. The view on the right shows the orientation of the fatty acid salts that was used to find their critical area. The view on the left shows a side view of the fatty acids in the absence of amine to emphasize the curved structures of the cis-fatty acids. The cis-fatty acid salts had larger critical areas than the saturated and trans-fatty acid salts they were not completely eclipsed by the triisobutylamine. The omega ends of the cis-fatty acids were not eclipsed by triisobutylamine, and these ends were “hooks” that increased the critical areas of the cis-fatty acid salts.

It is important to note that there are other methods to measure critical areas. For instance, we defined the critical area as possessing a rectangular shape, but other shapes (i.e. sphere, square, oval, etc) can also be used and will give different values for the critical areas. The variation of the critical area depending on the method of its measurement is an important reason why many nanofiltration membranes use a molecular weight cutoff rather than a critical area cutoff. Although the absolute value for the critical areas may be debatable, it was clear from FIG. 5 that cis-fatty acid salts had larger critical areas than the saturated and trans-fatty acid salts.

Separation and Isolation of Cis-Fatty Acids from Saturated and Trans-Fatty Acids.

The PDCPD membranes effectively retained cis-fatty acids, but at the completion of the experiment the upstream solvent contained a high concentration of saturated and trans-fatty acids due to how these experiments were conducted. The saturated and trans-fatty acids equilibrated between the solvent upstream and downstream of the membranes; at the end of the separations approximately 50% of these fatty acids were found in the upstream solvent with the cis-fatty acids. Thus, only approximately half of the saturated and trans-fatty acids were removed from the cis-fatty acids. This amount is much lower than would be desired for many applications.

To increase the purity of cis-fatty acids in the upstream solvent as well as the purity of the saturated and trans-fatty acids in the downstream solvent, a series of separations were completed using CH₂Cl₂ as the solvent. CH₂Cl₂ was chosen rather than the CH₂Cl₂/MeOH mixture due to the faster flux for fatty acid salts in CH₂Cl₂. In these experiments, the downstream solvent was periodically removed and replaced with fresh solvent. Replacing the downstream solvent lowered the concentration of the fatty acids in the downstream solvent which lowered the amount of fatty acid that permeated from the downstream solvent to the upstream solvent. This experiment was similar to a continuous extraction that is common in industrial applications.

In one experiment, a 1:1:2 mixture of stearic acid:oleic acid:triisobutylamine was added to 25 mL of CH₂CH₂ upstream of the membrane. On the downstream side 25 mL of CH₂Cl₂ was added. The upstream and downstream solvents were stirred for 24 h, and then the downstream solvent was removed and a fresh 25 mL of CH₂Cl₂ was added. The solvents were stirred for an additional 24 h, and then the downstream solvent was removed and replaced with fresh 25 mL of CH₂Cl₂. The solvents were stirred for 24 h and then the upstream and downstream solvents were removed. All of the downstream solvents were combined, the solvent evaporated, and the residual was analyzed by ¹H NMR spectroscopy. To extract any fatty acid within the PDCPD matrix, the membrane was extracted with CH₂Cl₂ and Et₃N twice as described in the experimental section. This solvent was combined with the upstream solvent and analyzed by ¹H NMR spectroscopy.

In this experiment, 90% of the stearic acid and only 13% of the oleic acid that were originally added to the apparatus were found in the downstream solvent. In contrast, 5% of the stearic acid and 86% of the oleic acid were found in the upstream solvent and membrane. For many applications purification of the cis-fatty acids is desired, and this experiment took a 1:1 molar ratio of stearic acid:oleic acid and to yield an isolated ratio of 1:17. Notably, 97% the fatty acids that were added to the apparatus were isolated at the end of the experiment. The fatty acids were not permanently trapped within the PDPCD membrane.

This experiment was repeated with four extractions rather than three as before, and the results were similar. Stearic acid (94%) and oleic acid (17%) were isolated from the downstream solvent, and stearic acid (3%) and oleic acid (81%) were isolated from the upstream solvent and membrane. Thus, the isolated ratio of stearic acid to oleic acid in the upstream solvent was 1:27. These results were very promising and demonstrated that the membranes could be used to separate oleic acid from stearic acid.

This experiment was repeated with a 1:1:1:1:4 molar ratio of stearic acid:oleic acid:linoleic acid:linolenic acid:triisobutylamine using CH₂Cl₂ as the solvent. This experiment was to simulate the separation of a small amount of a saturated fatty acid (stearic acid) from a mixture of three different cis-fatty acids. Four extractions were completed and the samples were analyzed as described in the supporting information. The downstream solvent possessed 92% of the stearic acid and only 5% of the cis-fatty acids that were originally added to the apparatus, but the upstream solvent and membrane had 4% of the stearic acid and 86% of the cis-fatty acids. Thus, most of the stearic acid was removed from the cis-fatty acids and the isolated ratio from the upstream solvent of stearic acid to cis-fatty acids was 1:22.

Use of Pressure to Increase Flux.

The cis-fatty acid salts were selectively retained while the saturated and trans-fatty acids salts readily permeated the membranes, but the values for flux were very low. No pressure was applied in these experiments, so the driving force for flux was based on differences in concentration of the molecules in solvent upstream and downstream of the membranes. Typical values for the flux of solvent through size selective membranes used in industry are around 10 L m⁻² h⁻¹, and these filtrations require less than an hour to complete. There are two important points to consider about how the industrial separations are completed and interpreted. First, they required the use of pressure on one side of the membrane or the separations were very slow. The use of pressure is not only acceptable, it is almost mandatory such that the filtrations are quick. Second, the values for flux are typically reported for the solvent rather than the molecule of interest (i.e. the product of a reaction). If the concentration of a product is approximately 100× lower than that of the solvent, then values for the flux of the products in a solvent are approximately 0.1 L m⁻² h⁻¹. Separations using PDCPD membranes required days to reach completion because no pressure was applied. An approximate value for the flux of a fatty acid through PDCPD membranes was 10⁻¹⁰ L m⁻² h⁻¹ which was far too slow for industrial applications.

Because the flux was very slow for the fatty acids, the use of pressure was studied. A metal vessel was used to apply pressure to solvent upstream of the membrane. A membrane was place horizontally within a metal vessel, and 100 mL of solvent with stearic acid, oleic acid, and triisobutylamine (1:1:2 molar ratio) were added to the solvent in the vessel. The reaction vessel was pressurized to 90 psi, and all of the solvent permeated within 20 min. It is important to note that that solvent was found on only one side of the membrane unlike in the experiments that did not use pressure. Initial experiments with mixtures of CH₂Cl₂ and methanol were unsuccessful due to the poor selectivity of the membrane (entries 1-3 in Table 6). Nearly all of the stearic acid salt (93-97%) permeated the membrane and was found in the solvent downstream of the membrane, but 77-80% of the oleic acid salt was found in the downstream solvent.

TABLE 6
Use of pressure to increase the flux through PDCPD membranes.

^{a}Amount permeated (%)

| Entry | Solvent | Stearic acid | Oleic acid |
|---|---|---|---|
| 1 | 90/10 CH₂Cl₂/MeOH | 97 | 78 |
| 2 | 75/25 CH₂Cl₂/MeOH | 93 | 80 |
| 3 | 60/40 CH₂Cl₂/MeOH | 95 | 77 |
| 4 | 40/60 toluene/hexanes | 94 | 36 |
| 5 | 35/65 toluene/hexanes | 99 | 22 |
| 6 | 30/70 toluene/hexanes | ^{b}0 | ^{b}0 |

^{a}These values refer to the fraction of each acid found in the downstream solvent relative to the amount of acid originally added to the upstream solvent.
^{b}The fatty acid salts did not permeate the membranes.

When a mixture of toluene and hexanes were studied, the difference in flux was much higher. At an optimal concentration of 35/65 (v/v) of toluene/hexanes, 99% of the stearic acid was found in the solvent downstream of the membrane but only 22% of the oleic acid was found in the downstream solvent. Some selectivity was lost compared to the experiments without pressure, but the time required for permeation was only 20 min which yielded a flux of for the solvent of 39 L m⁻² h⁻¹. This value for the flux was similar to values reported for membranes used in industry and represented a large improvement for the use of PDCPD membranes.

The use of multiple membranes to increase the selectivity for permeation of one molecule is commonly used in industrial labs, and this method was successful here too. To increase the separation of oleic acid from the stearic acid, the solvent downstream of the membrane was passed through a second PDCPD membrane using pressure. When the downstream solvent from entry 5 of Table 6 was passed through a second membrane, the amount of stearic acid that permeated was 96% of the original amount. In contrast, the amount of oleic acid that permeated decreased to only 7.5% of the original amount. Thus, the 1:1 molar ratio of stearic acid to oleic acid that was originally added was concentrated to a 13/1 ratio of stearic acid to oleic acid after passing through two PDCPD membranes.

This experiment was repeated to investigate the ratio of stearic acid to oleic acid upstream and downstream of the membranes. Briefly, a 1:1:2 molar ratio of stearic acid:oleic acid:triisobutylamine was passed through a PDCPD membrane using a 35/65 (v/v) toluene/hexanes mixture. The downstream solvent was then passed through a second PDCPD membrane under pressure. The downstream solvent after filtration through two PDCPD membranes had 95% of the original amount of stearic acid and only 7% of the original amount of oleic acid. The remainder of the oleic and stearic acid had permeated into the membranes and was retained within them. The membranes were removed from the apparatus and swollen in CH₂Cl₂ with Et₃N to extract the fatty acids. The CH₂Cl₂ extracts were then combined, the solvent was evaporated, and the distributions of products were analyzed by NMR spectroscopy. The recovery of oleic acid from the membrane was high (89% of the original amount added) and only 3% of the stearic acid was recovered from the membranes.

These results indicated a high level of success both in the overall recovery of the fatty acids and in the separation of saturated and cis-fatty acids. Nearly all of the stearic acid (98%) and oleic acid (96%) that was used at the beginning of the experiment was accounted for at the end. Most of the stearic acid (95%) was found downstream of the membrane and the ratio of stearic acid to oleic acid was 13.6/1 in the downstream solvent. In contrast, most of the oleic acid (89%) was retained by the membranes and the ratio of retained oleic acid to stearic acid was 30/1. These membranes were successful at separating a mixture of oleic acid/stearic acid.

Use of Pressure to Purify Fatty Acids Derived from Soybean Oil.

Soybean oil is one of the major sources of vegetable oils with over 35 million tons produced every year. Over 10 million tons of soybean oil are partially hydrogenated each year and used as food for humans and animals.^{4,6,7} The oil is partially hydrogenated because it has a high component of polyunsaturated cis-fatty acids (55% linoleic acid and 13% linolenic acid) that are prone to oxidation and lead to off-flavors or rancid food. The partial hydrogenation process introduces trans-fatty acids that were not present before, and these trans-fatty acids are highly undesired due to their negative effect on health. Furthermore, only a small fraction of soybean oil is used to produce fatty acids for industrial processes because of they are isolated as mixtures of fatty acids. Fatty acids may find increased applications in industry if a method to readily purify them was developed.

The separation of fatty acids derived from soybean oil was completed with PDCPD membranes under pressure. A mixture of 14% stearic acid, 18% oleic acid, 55% linoleic acid, and 13% linolenic acid was formulated from commercially available fatty acids. This mixture of fatty acids was added to 100 mL of hexanes:toluene (35/65, v/v) with one molar equivalent of triisobutylamine for every mole of fatty acid. This mixture was pressurized and allowed to permeate through two PDCPD membranes in series. The downstream solvent was removed and the residual was analyzed by ¹H NMR spectroscopy. The PDCPD membranes were soaked in CH₂Cl₂ with Et₃N to remove any fatty acids that had permeated into the matrix.

Nearly all of the stearic acid (94%) was found in the downstream solvent as expected based on its fast flux through PDCPD membranes (Table 7). Only 3% of oleic acid and 4% of the linolenic acid/linoleic acid permeated the membranes. The original mixture of fatty acids was only 14% by weight stearic acid, but after filtration through two PDCPD membranes the downstream solvent was 79% by weight stearic acid. Importantly, nearly all of the saturated fatty acid was removed from the unsaturated fatty acids. The fatty acids isolated from the PDCPD membranes contained less than 1% stearic acid. These membranes were very efficient at separating the saturated from unsaturated fatty acids.

TABLE 7
Separation of soybean oil through two PDCPD
membranes under pressure.

| | Initial amount | Downstream solvent | Upstream solvent |
|---|---|---|---|
| Molecule | (mmol) | (mmol) | (mmol) |
| Stearic acid | 0.426 | 0.401 | 0.022 |
| Oleic acid | 0.547 | 0.018 | 0.505 |
| Linolenic acid/ | 2.065 | 0.086 | 1.949 |
| Linoleic acid | | | |

It was hypothesized that the linolenic and linoleic acids could be selectively retained while oleic acid permeated the membranes. This hypothesis was based on the differences in shapes of the cis-fatty acid salts. Specifically, linoleic and linolenic acid had higher curvatures than oleic acid, and it was hypothesized that an oleic acid salt would possess a higher flux through PDCPD than the polyunsaturated acid salts (see FIG. 5 for the energy minimized structures of these fatty acids).

To test this hypothesis, the composition of fatty acids that permeated the two PDCPD membranes as shown in Table 7 were made into salts by the addition of one molar equivalent of Et₃N. The cis-fatty acid salts had very low flux when triisobutylamine was used, so smaller amines were investigated to find an amine salt where oleic acid permeated but linoleic and linolenic acid salts were retained. All of the fatty acid salts permeated the membrane after the application of pressure and no separation between oleic acid and the polyunsaturated fatty acid salts was found when Et₃N was used. This experiment was repeated with the same ratio of fatty acids salts that permeated the membranes as shown in Table 7 but with Pr₃N to form the salts. Here, pressure was applied across the membrane to reach fast values for the flux.

The oleic acid salt permeated the membrane and 0.33 mmoles were found in the downstream solvent, but only 0.43 mmoles of the polyunsaturated fatty acid salts permeated. The original mixture of fatty acids had a 1:3.8 ratio of oleic acid:polyunsaturated fatty acids but after permeation the ratio was 1:1.3. In the upstream solvent and the membrane, the ratio of oleic acid:polyunsaturated fatty acids was 1:7.3. Although this experiment did not lead to complete separation of oleic acid from linoleic and linolenic acid, it demonstrated that their salts possessed different flux.

Separation of Stearic and Oleic Acid Using Multiple Extractions.

A PDCPD membrane was added to the apparatus to study permeation. CH₂Cl₂ (25 mL) was added to the downstream side of the membrane and 25 mL of the same solvent was added to the upstream side of the membrane with 0.426 mmol of stearic acid, 0.426 mmol of oleic acid, and 0.852 mmol of triisobutylamine. Solvent on both sides of the membrane were stirred continuously at room temperature. After 24 and 48 h, the downstream solvent was replaced with fresh 25 mL of CH₂Cl₂. After 72 h, the downstream solvent was combined with the previous aliquots of downstream solvent. Also, the upstream solvent was removed and replaced with 25 mL of CH₂Cl₂ and 0.426 mmol of triethylamine to extract any fatty acid that was retained in the membrane. After 45 h, the solvent was removed and replaced with 25 mL of CH₂Cl₂ for a second recovery cycle. The downstream and upstream solvents were combined separately to determine the absolute amounts of stearic acid salt and oleic acid salt by ¹H NMR spectroscopy. The absolute amounts of the salts were found by the addition of known amounts of tetraethylene glycol to each aliquot.

The same experiment was repeated with four extractions of the downstream solvent. The remainder of the experiment was unchanged.

Use of Double Filtration to Increase the Selectivity for Permeation.

A PDCPD membrane was immersed in 30 mL of toluene:hexane (v/v, 35:65) solution for 15 min. After 15 min, the apparatus was assembled with the swollen membrane. Toluene:hexane (100 ml) was added to the upstream side of the membrane with 0.426 mmol of stearic acid, 0.426 mmol of oleic acid, and 0.852 mmol of triisobutylamine. The valve on the downstream side was opened. The pressure was increased to 90 psi in 10 min. After an induction period of a few hours where no solution permeated to the downstream side, the solution was collected on the downstream side in 15-20 min. The solvent was removed in vacuo. The residue was dissolved in 100 mL of toluene:hexane (v/v, 35:65) solution. Another PDCPD membrane was immersed in 30 mL of toluene:hexane (35:65) solution for 15 min. After 15 min, the apparatus was assembled with the swollen membrane. The solution prepared above was added to the upstream side of the membrane. The valve on the downstream side was opened. The pressure was increased to 90 psi in 10 min. After an induction period of a few hours where no solution permeated to the downstream side, the solution was collected on the downstream side in 15-20 min. The solvent was removed in vacuo. An aliquot of the residue was used to determine the absolute amounts of the stearic acid salt and oleic acid salts by ¹H NMR spectroscopy on the downstream side. The absolute amounts were found by the addition of known amounts of tetraethylene glycol to the aliquot. Each membrane from the two filtrations was immersed in 30 mL of CH₂Cl₂ with 0.426 mmol of triethylamine for 48 h to extract any fatty acid that was retained within the membrane. An aliquot was used to determine the absolute amounts of the stearic acid salt and oleic acid salt by ¹H NMR spectroscopy by the addition of known amounts of tetraethylene glycol.

Separation of Fatty Acids Derived from Soybean Oil Using PDCPD Membranes Under Pressure.

A PDCPD membrane was immersed in 30 mL of toluene:hexane (v/v, 35:65) solution for 15 min. After 15 min, the apparatus was assembled with the swollen membrane. Toluene:hexane (100 ml) was added to the upstream side of the membrane with 0.426 mmol of stearic acid, 0.547 mmol of oleic acid, 1.67 mmol of linoleic acid, 0.395 mmol of linolenic acid, and 3.038 mmol of triisobutylamine. The valve on the downstream side was opened. The pressure was increased to 90 psi in 10 min. After an induction period of a few hours where no solution permeated to the downstream side, the solution was collected on the downstream side in 15-20 min. The solvent was removed in vacuo. The residue was dissolved in 100 mL of toluene:hexane (v/v, 35:65) solution.

A second PDCPD membrane was immersed in 30 mL of toluene:hexane (35:65) solution for 15 min. After 15 min, the apparatus was assembled with the swollen membrane. The solution prepared above was added to the upstream side of the membrane. The valve on the downstream side was opened. The pressure was increased to 90 psi in 10 min. After an induction period of a few hours where no solution permeated to the downstream side, the solution was collected on the downstream side in 15-20 min. The solvent was removed in vacuo. An aliquot was used to determine the absolute amounts of the stearic acid salt, oleic acid, and polyunsaturated acid salts by ¹H NMR spectroscopy by the addition of known amounts of tetraethylene glycol. Each membrane from the two filtrations was immersed in 30 mL of CH₂Cl₂ and 2.5 mmol of triethylamine for 48 h. After the recovery of solvent from each membrane, the solvent was removed in vacuo. An aliquot was used to determine the absolute amounts of the salts by ¹H NMR spectroscopy by the addition of known amounts of tetraethylene glycol.

Separation of Cis-Fatty Acids Derived from Soybean Oil Using Triethylamine and Pressure (Table S1).

A PDCPD membrane was immersed in 30 mL of toluene:hexanes (v/v, 35:65) for 15 min. After 15 min, the membrane was added to a metal vessel. Toluene:hexane (v/v, 35:65, 100 ml) was added to the upstream side of the membrane with 0.016 mmol of stearic acid, 0.492 mmol of oleic acid, 1.477 mmol of linoleic acid, 0.395 mmol of linolenic acid, and 2.4 mmol of triethylamine. The valve on the downstream side was opened. The pressure was increased to 90 psi in 10 min. After an induction period of a few hours where no solution permeated to the downstream side, the solution was collected on the downstream side in 15-20 min. The aliquot was used to determine the absolute amounts of the stearic acid salt, oleic acid, and polyunsaturated acid salts by ¹H NMR spectroscopy. The absolute amounts of the salts were found by the addition of known amount of tetraethylene glycol to the aliquot.

TABLE S1
Separation of cis-fatty acids derived from
soybean oil using triethylamine

| | Initial Amount | Amount permeated |
|---|---|---|
| Molecule | (mmol) | (mmol) |
| Stearic acid | 0.016 | 0.012 |
| Oleic acid | 0.492 | 0.487 |
| Polyunsaturated acid | 1.872 | 1.72 |

Separation of Cis-Fatty Acids Derived from Soybean Oil Using Tripropylamine and Pressure (Table S2).

A PDCPD membrane was immersed in 30 mL of toluene:hexane (v/v, 35:65) for 15 min. After 15 min, the membrane was added to a metal vessel. Toluene:hexane (v/v, 35:65, 100 ml) was added to the upstream side of the membrane with 0.022 mmol of stearic acid, 0.505 mmol of oleic acid, 1.553 mmol of linoleic acid, 0.395 mmol of linolenic acid, and 2.475 mmol of tripropylamine. The valve on the downstream side was opened. The pressure was increased to 90 psi in 10 min. After an induction period of a few hours where no solution permeated to the downstream side, the solution was collected on the downstream side in 15-20 min. An aliquot was removed and used to determine the absolute amounts of stearic acid, oleic acid, and polyunsaturated acid salts by ¹H NMR spectroscopy. The absolute amounts of the salts were found by the addition of known amount of tetraethylene glycol to the aliquot.

The downstream solvent was permeated through the same membrane two more times using the same method. The downstream solvent was added to the reaction vessel containing the same membrane, the solvent was placed under pressure, and the solvent permeated. After each filtration, an aliquot was used to determine the absolute amounts of stearic acid, oleic acid, and polyunsaturated acid salts by ¹H NMR spectroscopy. The membrane was cut into pieces and immersed in 30 mL of CH₂Cl₂ and 2.5 mmol of triethylamine for 48 h. After recovery of solvent from the membrane, the solvent was removed in vacuo. An aliquot was used to determine the absolute amounts of the salts by ¹H NMR spectroscopy by the addition of known amounts of tetraethylene glycol.

TABLE S2
Separation of cis-fatty acids derived from
soybean oil using tripropylamine

| | Initial | | | | Membrane |
|---|---|---|---|---|---|
| | Amount | Cycle 1 | Cycle 2 | Cycle 3 | recovery |
| Molecule | (mmol) | (mmol) | (mmol) | (mmol) | (mmol) |
| Stearic acid | 0.022 | 0.018 | 0 | 0 | 0 |
| Oleic acid | 0.505 | 0.13 | 0.08 | 0.12 | 0.15 |
| Polyunsaturated | 1.949 | 0.14 | 0.10 | 0.19 | 1.4 |
| acid | | | | | |

Retention of Molecules Through PDCPD (FIG. 2).

Retention is defined as the percentage of a molecule that does not permeate through a membrane according to the equation below.

Retention (%)=(1−S_{d}/Sᵤ)×100

S_{d} is the concentration of molecule in the downstream solvent and Sᵤ is the concentration of molecule in the upstream solvent. In Table S3 we list 35 molecules whose retentions were known from prior work or were measured for this article. These molecules were used to plot the graphs in FIG. 2 in the main text.

TABLE S3
Critical area and retention of molecules through PDCPD.

| | | Molecular | Critical | | |
|---|---|---|---|---|---|
| | | Weight | Area | Retention | |
| Entry | Molecule | (gmol⁻¹) | (nm²) | (%) | Reference |
| 1 | triethylamine | 101 | 0.18 | 2 | 1 |
| 2 | quinuclidine | 111 | 0.21 | 7 | 1 |
| 3 | hexanoic acid | 116 | 0.067 | 6 | 1 |
| 4 | hexanediamine | 116 | 0.067 | 7 | 1 |
| 5 | tripropylamine | 143 | 0.32 | 13 | 1 |
| 6 | p-nitrobenzaldehyde | 151 | 0.06 | 0 | 1 |
| 7 | diphenylamine | 169 | 0.179 | 0 | 2 |
| 8 | triisobutylamine | 185 | 0.392 | 0 | 1 |
| 9 | tributylamine | 185 | 0.499 | 100 | 1 |
| 10 | hexadecane | 226 | 0.067 | 5 | 1 |
| 11 | triphenylmethane | 244 | 0.51 | 100 | 1 |
| 12 | triphenylamine | 245 | 0.492 | 100 | 2 |
| 13 | 2-tert-butyl-3-methyl-5-benzyl- | 246 | 0.36 | 30 | 1 |
| | 4-imidazolidinone | | | | |
| 14 | octadecene | 252 | 0.067 | 0 | 2 |
| 15 | triphenylphosphine | 262 | 0.612 | 100 | 1 |
| 16 | triphenylphosphine oxide | 278 | 0.612 | 100 | 1 |
| 17 | linolenic acid | 278 | 0.355 | 0 | 3 |
| 18 | tricyclohexylphosphine | 280 | 0.567 | 100 | 1 |
| 19 | linoleic acid | 280 | 0.338 | 0 | 3 |
| 20 | oleic acid | 282 | 0.212 | 0 | 3 |
| 21 | elaidic acid | 282 | 0.119 | 0 | 3 |
| 22 | vaccenic acid | 282 | 0.237 | 0 | 3 |
| 23 | petroselinic acid | 282 | 0.197 | 0 | 3 |
| 24 | stearic acid | 284 | 0.067 | 0 | 3 |
| 25 | binol | 286 | 0.505 | 100 | 1 |
| 26 | hexacosane | 366 | 0.067 | 30 | 3 |
| 27 | X-phos | 476 | 0.966 | 100 | 2 |
| 28 | tetracotriane | 479 | 0.067 | 69 | 3 |
| 29 | N,N′-bis(3,5-di-tert- | 492 | 0.79 | 100 | 1 |
| | butylsalicylidene)-1,2- | | | | |
| | cyclohexanediamine | | | | |
| 30 | methyl | 528 | 0.14 | 6 | 1 |
| | nonadecafluorodecanoate | | | | |
| 31 | nonaethylene glycol | 583 | 0.067 | 67 | 1 |
| | monododecyl ether | | | | |
| 32 | (R,R)-(−)-N,N′-bis(3,5-di-tert- | 662 | 1.15 | 100 | 1 |
| | butylsalicylidene)-1,2- | | | | |
| | cyclohexanediaminocobalt(II) | | | | |
| 33 | polystyrene | 800 | a | 88 | 3 |
| 34 | polystyrene | 1000 | a | 95 | 3 |
| 35 | polystyrene | 1260 | a | 100 | 3 |

a The critical area was ill-defined for polystyrene
1. Long, T.; Gupta, A.; Miller II, A. L.; Rethwisch, D.; Bowden, N. B. J. Mater. Chem. 2011, 21, 14265.
2. Gupta, A.; Long, T. R.; Rethwisch, D.; Bowden, N. B. Chem. Commun. 2011, 47, 10236.
3. Present work

### CONCLUSIONS

The conversion of a fatty acids to a fatty acid associated with a counterion (e.g. fatty acid salts) such as an amine led to the selective retention of a cis-fatty acid salts due to two effects. First, the counterion (e.g amines increased the critical areas of the fatty acids to the size range where PDCPD membranes could separate them. The free fatty acids were too small to be retained by the membranes, but the fatty acid salts were larger and in the size range where PDCPD membranes retain molecules. Second, the addition of the amines led to a larger difference in critical areas of the salts compared to the free fatty acids. The critical areas for the free fatty acids fell within a narrow range (0.067 to 0.36 nm²), but the critical areas for the fatty acid salts fell within a large range (0.38 to 1.27 nm²).

It was surprising and unexpected that the formation of a noncovalent, reversible interaction between a fatty acid and an amine led to a large difference in permeation. The hooks on the cis-fatty acids were distant from the amines which were the largest, bulkiest part of the salts. Furthermore, fatty acids have large numbers of C—C single bonds that rotate at room temperature and lead to a wide variety of different conformations, and critical areas, for each fatty acid. It would have been reasonable to assume that the flexibility of the fatty acids coupled with the distance between the hooks and the amine would have led to little or no effective difference in critical areas or flux between the fatty acid salts. Yet, the addition of amines led to a difference in critical areas and a significant difference in permeation.

The separation of fatty acids using membranes is an important advancement in this field. Over 140 million tons of vegetable oils are produced each year, but over 96% of it is used for food, feed for animals, or biodiesel. There are surprisingly few other industrial applications of fatty acids despite their low cost and abundance. The reason for the limited industrial applications to turn fatty acids into more valuable materials is that they are isolated as mixtures and it is not possible to separate these mixtures into individual components on a large, industrial scale. Currently, any industrial application of fatty acids requires using a mixture of fatty acids (such as those derived from soybean oils). The method described herein to separate fatty acids using membranes is an important advance because membranes are widely used in industry and can be used to purify large quantities of a molecule. This represents a new approach to solving a critical problem.

All publications, patents, and patent documents discussed herein are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for separating a mixture of two or more fatty acids associated with counterions comprising contacting a membrane with a first mixture comprising two or more different fatty acids associated with counterions, so that the mixture is fractionated into a permeate comprising one or more different fatty acids associated with counterions and a retentate comprising one or more different fatty acids associated with counterions, wherein at least one of the permeate or retentate is enriched in one or more different fatty acids associated with counterions.

2. The method of claim 1 wherein the permeate is enriched in one or more different fatty acids associated with counterions.

3. The method of claim 1 wherein the retentate is enriched in one or more different fatty acids associated with counterions.

4. The method of claim 1 wherein the permeate is enriched in one or more different fatty acids associated with counterions and the retentate is enriched in one or more different fatty acids associated with counterions.

5. The method of any one of claims 1-4 wherein the membrane is an organic solvent nanofiltration membrane.

6. The method of claim 5 wherein the organic solvent nanofiltration membrane comprises polydicyclopentadiene, polyimide, polyaniline or polyacrylate.

7. The method of claim 5 wherein the organic solvent nanofiltration membrane comprises polydicyclopentadiene.

8. The method of claim 7 wherein the organic solvent nanofiltration membrane comprises a highly crosslinked polydicyclopentadiene matrix.

9. The method of claim 8 wherein the ratio of crosslinked double bonds to uncrosslinked double bonds in the highly cross-linked polydicyclopentadiene matrix is at least about 2:3.

10. The method of claim 8 wherein the ratio of crosslinked double bonds to uncrosslinked double bonds in the highly cross-linked polydicyclopentadiene matrix is at least about 3:2.

11. The method of claim 8 wherein the ratio of crosslinked double bonds to uncrosslinked double bonds in the highly cross-linked polydicyclopentadiene matrix is at least about 4:1.

12. The method of any one of claims 1-11 wherein the membrane is a part of an assembly that comprises two or more membranes.

13. The method of any one of claims 1-11 wherein membrane is part of a spiral wound module.

14. The method of any one of claims 1-11 wherein the membrane separates molecules based on their cross-sectional areas.

15. The method of any one of claims 1-14 wherein each fatty acid of the first mixture, permeate and retentate is associated with one counterion and wherein the counterions are identical.

16. The method of any one of claims 1-15 wherein the counterion has a critical area that allows one or more different fatty acids associated with the counterion of the first mixture to permeate the membrane at a higher rate than one or more different fatty acids associated with the counterion of the first mixture so that the permeate is enriched in one or more different fatty acids associated with the counterion.

17. The method of any one of claims 1-16 wherein the counterion has a critical area that impedes the permeation of one or more different fatty acids associated with the counterion of the first mixture through the membrane so that the retentate is enriched in one or more fatty acids associated with the counterion.

18. The method of any one of claims 1-17 wherein the counterion has a critical area of about 0.38±0.08 nm².

19. The method of any one of claims 1-17 wherein the counterion has a critical area of 0.38±0.04 nm².

20. The method of any one of claims 1-17 wherein the counterion has a critical area of 0.38±0.02 nm².

21. The method of any one of claims 1-20 wherein counterion is a positively charged amine.

22. The method of claim 21 wherein the positively charged amine is:
(a)⁺NR₄ wherein each R is independently hydrogen, optionally substituted (C₁-C₈)alkyl, optionally substituted (C₂-C₈)alkenyl, optionally substituted (C₂-C₈)alkynyl or optionally substituted (C₃-C₈)cycloalkyl or
(b) an optionally substituted cyclic amine wherein at least one of the nitrogen atoms which comprise the cyclic amine is positively charged.

23. The method of claim 21 wherein the positively charged amine is ⁺NR₄ wherein each R is independently hydrogen, optionally substituted (C₁-C₈)alkyl, optionally substituted (C₂-C₈)alkenyl, optionally substituted (C₂-C₈)alkynyl or optionally substituted (C₃-C₈)cycloalkyl.

24. The method of any one of claims 1-20 wherein the counterion is a tetralkylammonium, trialkylammonium, dialkylammonium or monalkylammonium.

25. The method of any one of claims 1-24 wherein the counterion is a trialkylammonium.

26. The method of any one of claims 1-24 wherein the counterion is triisobutylammonium.

27. The method of any one of claims 1-26 wherein the first mixture comprises:
a) at least one cis-fatty acid associated with a counterion and
b) at least one trans-fatty acid associated with a counterion or at least one saturated fatty acid associated with a counterion.

28. The method of any one of claims 1-26 wherein the first mixture comprises at least one cis-fatty acid associated with a counterion and at least one saturated fatty acid associated with a counterion.

29. The method of any one of claims 1-26 wherein the first mixture comprises two or more different cis-fatty acids associated with counterions.

30. The method of any one of claims 1-29 wherein the permeate is enriched in at least one fatty acid associated with a counterion wherein the fatty acid associated with a counterion has critical area of less than or equal to about 0.42 nm².

31. The method of any one of claims 1-29 wherein the permeate is enriched in at least one fatty acid associated with a counterion wherein the fatty acid associated with a counterion has critical area of less than or equal to about 0.38 nm².

32. The method of any one of claims 1-29 wherein the permeate is enriched in at least one fatty acid associated with a counterion wherein the fatty acid has critical area of less than or equal to about 0.12 nm².

33. The method of any one of claims 1-29 wherein the permeate is enriched in at least one fatty acid associated with a counterion wherein the fatty acid has critical area of less than or equal to about 0.07 nm².

34. The method of any one of claims 1-29 wherein the permeate is enriched in at least one fatty acid associated with a counterion wherein the fatty acid associated with a counterion has critical area of less than or equal to about 0.65 nm².

35. The method of any one of claims 1-29 wherein the permeate is enriched in at least one fatty acid associated with a counterion wherein the fatty acid associated with a counterion has critical area of less than or equal to about 0.59 nm².

36. The method of any one of claims 1-29 wherein the permeate is enriched in at least one fatty acid associated with a counterion wherein the fatty acid has critical area of less than or equal to about 0.23 nm².

37. The method of any one of claims 1-29 wherein the permeate is enriched in at least one fatty acid associated with a counterion wherein the fatty acid has critical area of less than or equal to about 0.21 nm².

38. The method of any one of claims 1-33 wherein the permeate is enriched in at least one trans-fatty acid associated with a counterion or one saturated fatty acid associated with a counterion.

39. The method of any one of claims 1-33 wherein the permeate is enriched in at least one saturated fatty acid associated with a counterion.

40. The method of any one of claims 1-37 wherein the permeate is enriched in at least one cis-fatty acid associated with a counterion.

41. The method of any one of claims 1-40 wherein the retentate is enriched in at least one fatty acid associated with a counterion wherein the fatty acid associated with a counterion has critical area of greater than or equal to about 0.53 nm².

42. The method of any one of claims 1-40 wherein the retentate is enriched in at least one fatty acid associated with a counterion wherein the fatty acid associated with a counterion has critical area of greater than or equal to about 0.59 nm².

43. The method of any one of claims 1-40 wherein the retentate is enriched in at least one fatty acid associated with a counterion wherein the fatty acid associated with a counterion has critical area of greater than or equal to about 0.83 nm².

44. The method of any one of claims 1-40 wherein the retentate is enriched in at least one fatty acid associated with a counterion wherein the fatty acid has critical area of greater than or equal to about 0.19 nm².

45. The method of any one of claims 1-40 wherein the retentate is enriched in at least one fatty acid associated with a counterion wherein the fatty acid has critical area of greater than or equal to about 0.21 nm².

46. The method of any one of claims 1-40 wherein the retentate is enriched in at least one fatty acid associated with a counterion wherein the fatty acid has critical area of greater than or equal to about 0.29 nm².

47. The method of any one of claims 1-46 wherein the retentate is enriched in at least one cis-fatty acid associated with a counterion.

48. The method of any one of claims 1-26 wherein the first mixture comprises soybean oil wherein the fatty acid components of the soybean oil are associated with counterions.

49. The method of any one of claims 1-26 wherein the first mixture comprises palmitic acid, stearic acid, linolenic acid, oleic acid and linoleic acid each associated with a counterion.

50. The method of claim 48 or claim 49 wherein the permeate is enriched in palmitic acid and stearic acid each associated with a counterion.

51. The method of any one of claims 48-50 wherein the retentate is enriched in linolenic acid, oleic acid and linoleic acid each associated with a counterion.

52. The method of any one of claims 1-26 wherein the first mixture comprises linolenic acid, oleic acid and linoleic acid each associated with a counterion.

53. The method of claim 52 wherein the permeate is enriched in oleic acid associated with a counterion.

54. The method of claim 52 or claim 53 wherein the retentate is enriched in linolenic acid and linoleic acid each associated with a counterion.

55. The method of claim 52 or claim 53 wherein the retentate is enriched in linolenic acid associated with a counterion.

56. The method of claim 52 or claim 53 wherein the retentate is enriched in linoleic acid associated with a counterion.

57. The method of any one of claim 1-56 wherein the permeate is removed one or more times during the separation.

58. The method of any one of claim 1-56 wherein the permeate is removed one or more times during the separation and replaced with a solvent.

59. The method of any one of claim 1-56 wherein the permeate is removed continuously.

60. The method of any one of claim 1-59 wherein the first mixture comprises a solvent.

61. The method of claim 60 wherein the solvent comprises one or more protic or aprotic organic solvents.

62. The method of claim 60 wherein the solvent comprises toluene, hexane, methanol or methylene chloride or mixtures thereof.

63. The method of claim 60 wherein the solvent comprises methanol and methylene chloride.

64. The method any one of claim 1-63 wherein pressure is applied to the first mixture to increase the flux of the first mixture through the membrane.

65. A mixture comprising two or more different fatty acids wherein the fatty acids are associated with a counterion wherein the counterion has a critical area of 0.38 nm²±0.04 nm².

66. The mixture of claim 65 wherein the counterion has a critical area of 0.38±0.02 nm².

67. The mixture of claim 65 wherein the counterion has a critical area of about 0.38 nm².

68. The mixture of claim 65 wherein the counterion is triisobutylammonium.

69. The mixture of any one of claims 65-68 wherein the each fatty acid of the mixture is associated with one counterion and wherein the counterions are identical.

70. The mixture of any one of claims 65-69 which comprises:
a) at least one cis-fatty acid associated with a counterion and
b) at least one trans-fatty acid associated with a counterion or at least one saturated fatty acid associated with a counterion.

71. The mixture of any one of claims 65-69 which comprises two or more cis-fatty acids associated with counterions.

72. The mixture of any one of claims 65-69 which comprises palmitic acid, stearic acid, linolenic acid, oleic acid and linoleic acid each associated with a counterion.

73. The mixture of any one of claims 65-69 which comprises linolenic acid, oleic acid and linoleic acid each associated with a counterion.
